(19) United States
(12) Patent Application Publication
(10) Pub. No.: US 2023/0398193 A1
(43) Pub. Date: Dec. 14, 2023

(54) ASPARAGINASE PROTEINS

(71) Applicant: Flagship Pioneering Innovations VI, LLC, Cambridge, MA (US)

(72) Inventors: Meghan Whitney FRANKLIN, Portland, ME (US); Gevorg GRIGORYAN, Lexington, MA (US); Daniel Solomon LEVENTHAL, Medford, MA (US)

(21) Appl. No.: 18/032,860

(22) PCT Filed: Oct. 19, 2021
(86) PCT No.: PCT/US2021/071931

§ 371 Date: Apr. 20, 2023

Related U.S. Application Data
(60) Provisional application No. 63/093,974, filed on Oct. 20, 2020.

(51) Int. Cl. A61K 38/50; C12N 9/82; A61P 35/00; C12N 15/63

(52) U.S. Cl. CPC A61K 38/50; C12N 9/82; A61P 35/00; C12N 15/63; C12Y 305/01001

(57) ABSTRACT

The disclosure provides, in various embodiments, polypeptides that upon administration to a mammalian subject, elicit a reduced immunogenicity in the subject, relative to a bacterial asparaginase. The disclosure also provides, in various embodiments, fusion proteins comprising one or more of the polypeptides, polynucleotides encoding the polypeptides, vectors and host cells suitable for expressing the polypeptides, methods for treating a disease (e.g., cancer) in mammalian subject (e.g., a human), and methods for reducing a level of asparagine or asparagine-containing product in a biological fluid from a mammalian subject.

### RELATED APPLICATION(S)

This application claims the benefit of U.S. Provisional Application No. 63/093,974, filed on Oct. 20, 2020. The entire teachings of the above application are incorporated herein by reference.

### INCORPORATION BY REFERENCE OF MATERIAL IN ASCII TEXT FILE

This application incorporates by reference the Sequence Listing contained in the following ASCII text file being submitted concurrently herewith:
a) File name: 57081031001_Sequence_Listing.txt; created Oct. 19, 2021, 42,461 bytes in size.

### BACKGROUND

Neutralizing antibodies (nAbs) or anti-drug antibodies (ADAs) limit the effectiveness of therapeutic proteins, especially those that must be administered over an extended period. E. coli asparaginase (ASN), a therapeutic that exhibits high rates of ADA development following administration in an oncology setting, is utilized for the treatment of several hematologic malignancies, including as a front-line therapy for acute lymphoblastic leukemia (ALL). Approximately 30% of ALL patients will development hypersensitivity to E. coli ASN treatment and be placed on Erwinia ASN as a second line therapy.

### SUMMARY

There is a critical need to develop asparaginase polypeptides that can evade an existing neutralizing antibody response upon administration to a mammalian subject, can elicit a reduced immunogenicity in the mammalian subject, or a combination thereof, relative to, for example a bacterial asparaginase.

The disclosure provided herein is based, at least in part, on Applicant's ability to reengineer bacterial asparaginases to, for example, avoid existing anti-drug antibodies. Accordingly, the disclosure generally relates to compositions (e.g., polypeptides, pharmaceutical compositions) comprising engineered bacterial asparaginases that elicit reduced immunogenicity in mammalian subjects, relative to a parental non-engineered (e.g., endogenous or wild type) bacterial asparaginase, and methods of using said compositions.

In one aspect, the disclosure provides de-immunized asparaginase polypeptides that, upon administration to a mammalian subject, elicit a reduced immunogenicity in the subject, relative to a bacterial asparaginase.

In some embodiments, the polypeptide comprises an amino acid sequence that is at least about 30% identical to the amino acid sequence of E. coli asparaginase (SEQ ID NO:1), Erwinia asparaginase (SEQ ID NO:2), or a combination thereof. In some embodiments, the polypeptide comprises at least one amino acid substitution, relative to the amino acid sequence of E. coli asparaginase (SEQ ID NO:1), Erwinia asparaginase (SEQ ID NO:2), or a combination thereof.

In some embodiments, the polypeptide comprises an amino acid sequence that is at least about 75% identical to the amino acid sequence of E. coli asparaginase (SEQ ID NO:1). In some embodiments, the polypeptide comprises about 5-60 amino acid substitutions, relative to the amino acid sequence of E. coli asparaginase (SEQ ID NO:1).

In some embodiments, the polypeptide comprises an amino acid substitution at a position corresponding to one or more of positions:
a) 54, 201, 202, 232, 263 or 311 of SEQ ID NO:1;
b) 2, 37, 43, 54, 64, 68, 75, 139, 140, 143, 196, 199, 202, 209, 210, 225, 252, 255, 259, 263, 312, 314, 317, 318, 321, 324 or 325 of SEQ ID NO:1;
c) 2, 33, 34, 37, 54, 64, 68, 71, 139, 143, 196, 199, 200, 202, 207, 209, 225, 252, 259, 262, 263, 312, 317, 318, 321, 324 or 325 of SEQ ID NO:1;
d) 1, 2, 3, 34, 37, 38, 47, 54, 64, 68, 75, 139, 140, 143, 196, 199, 202, 209, 225, 233, 259, 262, 263, 312, 317, 318, 321, 324 or 325 of SEQ ID NO:1;
e) 1, 2, 37, 49, 54, 64, 68, 71, 75, 139, 140, 143, 196, 199, 200, 202, 207, 209, 210, 225, 252, 255, 259, 262, 263, 312, 317, 318, 321, 324, 325 of SEQ ID NO:1;
f) 33, 37, 38, 54, 64, 68, 75, 139, 140, 143, 199, 200, 202, 207, 209, 252, 255, 263, 287, 312, 314, 317, 318, 321, 324, 325 of SEQ ID NO:1;
g) 37, 54, 68, 75, 140, 143, 199, 200, 225, 263, 312 or 314 of SEQ ID NO:1;
h) 1, 2, 3, 33, 34, 36, 37, 38, 43, 47, 49, 54, 64, 68, 72, 75, 76, 78, 79, 80, 106, 138, 139, 140, 142, 143, 144, 192, 196, 199, 200, 201, 202, 208, 209, 224, 225, 229, 232, 233, 252, 255, 256, 258, 259, 263, 265, 287, 288, 311, 312, 314, 316, 317, 318, 321, 324 or 325 of SEQ ID NO:1;
i) 37, 50, 54, 64, 68, 75, 140, 196, 199, 200, 202, 209, 225, 252, 255, 259, 262, 263, 287, 312, 314, 317, 318, 321, 324 or 325 of SEQ ID NO:1; or
j) 1, 2, 3, 33, 34, 37, 38, 43, 47, 49, 50, 52, 54, 64, 68, 71, 75, 78, 104, 106, 139, 143, 199, 207, 209, 210, 225, 233, 252, 255, 259, 263, 288, 312, 317, 318, 321, 324 or 325 of SEQ ID NO:1.

In some embodiments, the polypeptide comprises an amino acid substitution at a position corresponding to the position in SEQ ID NO:1 selected from:
a) V54K, T201S, P202E, V232I, T263D or T311I, or a combination thereof;
b) P2K, N37K, K43A, V54K, N64E, T68K, T75D, K139Q, A140R, N143G, K196P, S199P, P202E, N209T, E210S, D225V, S252E, D255K, T259E, T263N, Q312K, K314N, Q317E, Q318E, Q321R, N324D or Q325T, or a combination thereof,
c) P2K, E33D, N34K, N37E, V54K, N64E, T68K, K71L, K139P, N143G, K196P, S199L, D200A, P202E, K207S, N209T, D225V, S252E, T259K, K262E, T263D, Q312K, Q317E, Q318E, Q321K, N324D or Q325T, or a combination thereof,
d) L1I, P2K, N3K, N34E, N37E, A38R, N47E, V54M, N64E, T68K, T75E, K139Q, A140R, N143G, K196P, S199P, P202E, N209T, D225S, D233E, T259K, K262E, T263D, Q312K, Q317E, Q318E, Q321R, N324D or Q325T, or a combination thereof,
e) L1I, P2K, N37K, K49R, V54K, N64E, T68K, K71E, T75K, K139Q, A140R, N143G, K196L, S199P, D200L, P202E, K207S, N209D, E210A, D225A, S252E, D255Q, T259Q, K262A, T263D, Q312K, Q317E, Q318E, Q321E, N324D or Q325R, or a combination thereof;
f) E33D, N37A, A38L, V54K, N64E, T68K, T75D, K139E, A140R, N143G, S199P, D200L, P202E, K207N, N209T, S252L, D2551, T263N, A287E, Q312K, K314N, Q317R, Q318E, Q321E, N324D or Q325T, or a combination thereof,
g) N37K, V54K, T68R, T75D, A140R, N143G, S199P, D200L, D225S, T263N, Q312K or K314N, or a combination thereof,
h) L1K, P2K, N3K, E33D, N34E, V36I, N37A, A38R, K43A, N47E, K49D, V54M, N64E, T68K, K72R, T75E, D76L, D78E, K79Q, T80Y, D106S, D138H, K139P, A140E, A142K, N143G, R144Q, T192Q, K196P, S199L, D200A, T201S, P202E, L208I, N209E, S224F, D225V, K229E, V232I, D233K, S252E, D255K, T256A, A258K, T259E, T263N, T265I, A287E, K288Q, T311V, Q312K, K314N, P316R, Q317R, Q318E, Q321R, N324D or Q325T, or a combination thereof,
i) N37A, G50V, V54K, N64E, T68K, T75D, A140R, K196L, S199L, D200E, P202E, N209D, D225V, S252E, D255K, T259E, K262R, T263R, A287E, Q312K, K314N, Q317R, Q318E, Q321E, N324R or Q325R, or a combination thereof; or
j) L1M, P2K, N3K, E33D, N34E, N37A, A38G, K43A, N47D, K49T, G50A, Q52E, V54R, N64E, T68E, K71R, T75E, D78E, K104T, D106N, K139D, N143G, S199K, K207G, N209T, E210S, D225S, D233E, S252E, D255E, T259E, T263N, K288A, Q312L, Q317A, Q318A, Q321A, N324D or Q325E, or a combination thereof.

In some embodiments, the polypeptide comprises a conservative substitution, a highly conservative substitution or no substitution, at the positions corresponding to each of positions 12, 25, 89, 90, 162 and 283 of SEQ ID NO:1. In some embodiments, the polypeptide does not comprise a substitution at the positions corresponding to each of positions 12, 25, 89, 90, 162 and 283 of SEQ ID NO:1.

In some embodiments, the polypeptide comprises a conservative substitution, a highly conservative substitution or no substitution, at the positions corresponding to each of positions 23, 41, 60, 63, 94, 121, 122, 123, 151, 156, 163, 164, 165, 167, 175, 176, 181, 184, 186, 218, 222, 248, 250, 251, 272, 275, 277, 281 and 300 of SEQ ID NO:1. In some embodiments, the polypeptide does not comprise a substitution at the positions corresponding to each of positions 23, 41, 60, 63, 94, 121, 122, 123, 151, 156, 163, 164, 165, 167, 175, 176, 181, 184, 186, 218, 222, 248, 250, 251, 272, 275, 277, 281 and 300 of SEQ ID NO:1.

In some embodiments, the polypeptide does not comprise a T→V or T→Q substitution at the position corresponding to the T residue at position 192 of SEQ ID NO:1. In some embodiments, the polypeptide does not comprise a substitution at the position corresponding to position 192 of SEQ ID NO:1.

In some embodiments, the polypeptide does not comprise a substitution of the T residue at the position corresponding to position 80 of SEQ ID NO:1 with an aromatic amino acid. In some embodiments, the polypeptide does not comprise a substitution at the position corresponding to position 80 of SEQ ID NO:1.

In some embodiments, the polypeptide comprises an amino acid sequence set forth in any one of SEQ ID Nos:4-13, or a variant thereof comprising an amino acid sequence that is at least 50% identical to the amino acid sequence of any one of SEQ ID NOs:4-13, or a combination thereof. In some embodiments, the polypeptide comprises one or more amino acid substitutions, relative to the amino acid sequence of any one of SEQ ID NOs:4-13, or a combination thereof. In some embodiments, the polypeptide comprises the amino acid sequence of SEQ ID NO: 4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12 or SEQ ID NO:13.

In some embodiments, the polypeptide is conjugated to a heterologous moiety.

In other aspects, the disclosure provides a fusion protein comprising a polypeptide disclosed herein.

In other aspects, the disclosure provides a polynucleotide comprising a sequence encoding a polypeptide disclosed herein.

In other aspects, the disclosure provides an expression vector comprising a polynucleotide disclosed herein.

In other aspects, the disclosure provides a host cell comprising the polynucleotide or an expression vector disclosed herein.

In other aspects, the disclosure provides a composition comprising a polypeptide, fusion protein, polynucleotide, expression vector or host cell disclosed herein. In some embodiments, the further comprises one or more pharmaceutically acceptable excipients, diluents, or carriers.

In other aspects, the disclosure provides a method of reducing a level of asparagine or asparagine-containing product in a biological fluid (e.g., from a mammalian subject), comprising contacting the biological fluid with an effective amount of a polypeptide, fusion protein or composition disclosed herein.

In other aspects, the disclosure provides a method of treating a mammalian subject in need thereof, comprising administering an effective amount of a polypeptide, fusion protein or composition disclosed herein to the mammalian subject. In some embodiments, the mammalian subject has cancer.

In other aspects, the disclosure provides a method of treating cancer in a mammalian subject in need thereof, comprising administering an effective amount of a polypeptide, fusion protein or composition disclosed herein to the mammalian subject.

In some embodiments, the cancer is a hematological cancer. In some embodiments, the hematological cancer is acute lymphoblastic leukemia (ALL), non-Hodgkin's lymphoma or acute myeloid leukemia (AML).

In some embodiments, the mammalian subject has an immune response to one or more bacterial asparaginases. In some embodiments, the mammalian subject is at risk of developing an immune response to one or more bacterial asparaginases.

In some embodiments, the mammalian subject is a human.

In some embodiments, the method further comprises administering a second therapeutic agent to the mammalian subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A and 1B depict a multiple sequence alignment for resurfaced ASNs (SEQ ID NOs:4-13) compared to the parental E. coli ASN protein (SEQ ID NO: 1). The active site residues are shaded in green and interfacial positions are shaded in blue.

FIG. 2 is a graph plotting expression versus activity for E. coli asparaginase and 14 resurfaced asparaginase variants. The ten resurfaced ASNs selected for large scale production and testing are labeled above. All other resurfaced ASNs are shown in the bottom left corner and failed to express and/or exhibit ASN activity above background controls.

FIGS. 3A and 3B are graphs depicting the results of ELISA assays to determine relative binding of rabbit and mouse anti-ASN ADAs against ASN variants and controls. Anti-rabbit- or anti-mouse-IgG-HRP were used to detect binding of polyclonal ADAs (OD at 450 nM). Highest-to-lowest ranked binding (top-to-bottom) is shown at the right of each plot.

FIGS. 4A and 4B are graphs depicting relative binding (OD_{450 nm}) to rabbit and mouse ADAs as a function of hamming distance (number of mutations relative to the parental E. coli ASN) for each resurfaced ASN. A linear regression was performed to establish a best-fit curve between all data points with the resulting R2 shown.

FIG. 5 is a graph depicting percent binding (relative to WT E. coli ASN control) to human ADAs from patient serum or plasma for each resurfaced ASN and the Erwinia ASN negative control. Each dot represents a measurement from an individual donor sample, with box and whisker plots showing mean, min and max for each test article. An unpaired, two tailed t test was performed to measure statistical significance for each test article compared to WT E. coli ASN control (*=P<0.05, **=P<0.005, ***=P<0.0005, ****=P<0.00005).

### DETAILED DESCRIPTION

The disclosure disclosed herein is based, at least in part, on Applicant's ability to reengineer bacterial asparaginases to, for example, avoid existing anti-drug antibodies. A description of example embodiments follows.

### Polypeptides

The disclosure disclosed herein is based, at least in part, on the discovery of polypeptides that maintain the functional properties of bacterial asparaginase proteins and are capable of evading pre-existing neutralizing responses or adverse events elicited by bacterial asparaginase proteins, and/or have reduced immunogenicity compared to bacterial asparaginase proteins, when administered to a mammalian subject. Accordingly, the disclosure generally relates to compositions (e.g., de-immunized asparaginases, pharmaceutical compositions comprising de-immunized asparaginases) and methods that are useful for reducing levels of asparagine in a mammalian subject in need thereof.

Accordingly, provided herein are de-immunized asparaginase polypeptides that exhibit reduced immunogenicity, relative to a bacterial asparaginase, upon administration to a mammalian subject in need thereof.

As used herein, the term “polypeptide” “peptide” or “protein” denotes a polymer of at least two amino acids covalently linked by an amide bond, regardless of length or post-translational modification (e.g., glycosylation or phosphorylation). A protein, peptide or polypeptide can comprise any suitable L- and/or D-amino acid, for example, common α-amino acids (e.g., alanine, glycine, valine), non-α-amino acids (e.g., β-alanine, 4-aminobutyric acid, 6-aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and methods for adding or removing protecting groups are known in the art and are disclosed in, for example, Green and Wuts, “Protecting Groups in Organic Synthesis,” John Wiley and Sons, 1991. The functional groups of a protein, peptide or polypeptide can also be derivatized (e.g., alkylated) or labeled (e.g., with a detectable label, such as a fluorogen or a hapten) using methods known in the art. A protein, peptide or polypeptide can comprise one or more modifications (e.g., amino acid linkers, acylation, acetylation, amidation, methylation, terminal modifiers (e.g., cyclizing modifications), N-methyl-α-amino group substitution), if desired. In addition, a protein, peptide or polypeptide can be an analog of a known and/or naturally-occurring peptide, for example, a peptide analog having conservative amino acid residue substitution(s).

As used herein, the term “de-immunized asparaginase polypeptide” refers to a polypeptide having (1) an asparaginase activity, which is the ability to break down asparagine, such as L-asparagine, e.g., to aspartic acid and ammonium; and (2) a reduced immunogenicity in a mammalian subject, such as a human, relative to a bacterial asparaginase, such as E. coli asparaginase (SEQ ID NO:1) or Erwinia asparaginase (SEQ ID NO:2). In some embodiments, “reduced immunogenicity” refers to a de-immunized asparaginase polypeptide that elicits a lesser (e.g., in magnitude, in duration) immune response (e.g., neutralizing antibody response and/or anti-drug antibody response) or adverse event, relative to a bacterial asparaginase, upon administration to a mammalian subject. In some embodiments, a de-immunized asparaginase polypeptide has reduced binding to neutralizing antibodies and/or anti-drug antibodies relative to a bacterial asparaginase (e.g., neutralizing antibodies and/or anti-drug antibodies can have a lower binding affinity for the de-immunized asparaginase compared to a bacterial asparaginase). The ability of a de-immunized asparaginase polypeptide to elicit a lesser immune response relative to a bacterial asparaginase can be assessed e.g., by the methods of Example 3 or Example 4, infra.

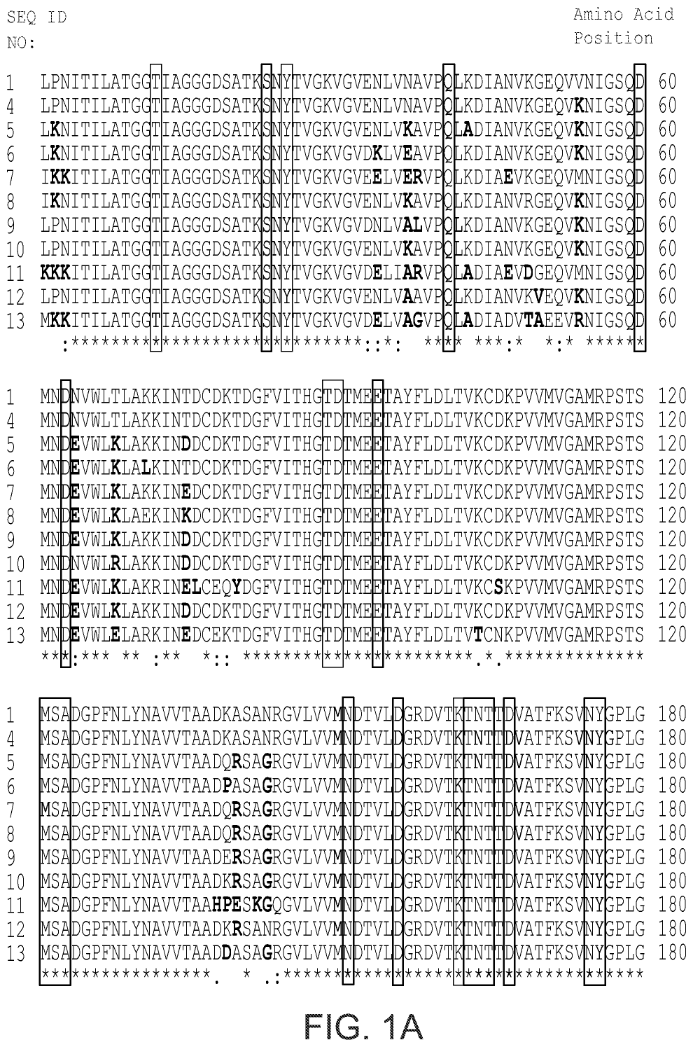

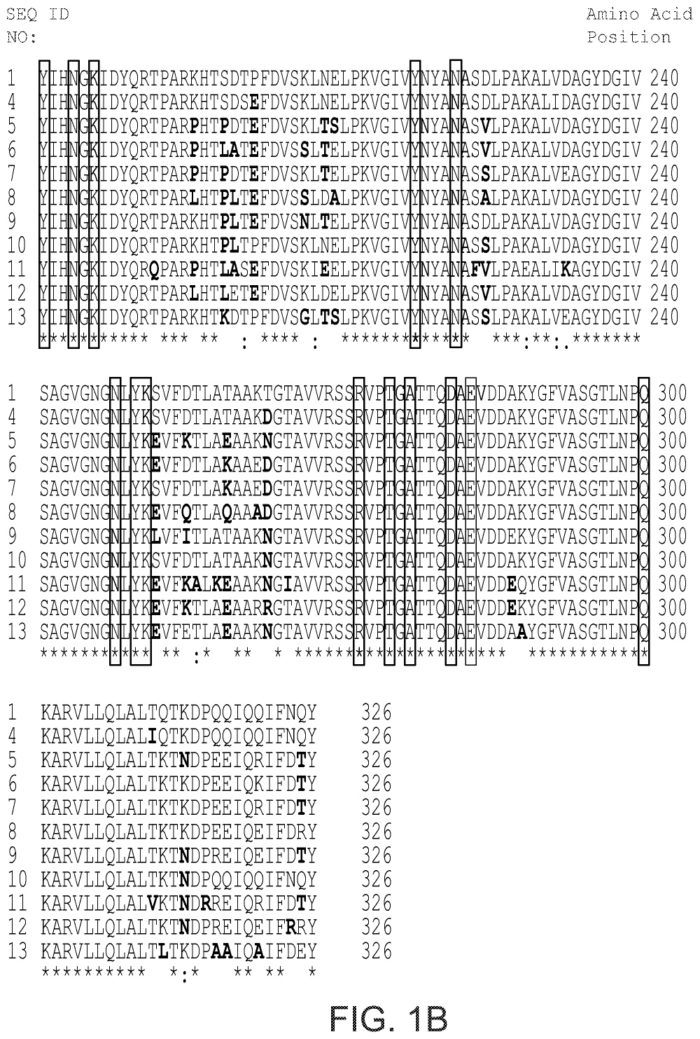

In some embodiments, a polypeptide of the disclosure comprises an amino acid sequence that is a variant of a bacterial asparaginase. In some embodiments, the bacterial asparaginase is E. coli asparaginase (SEQ ID NO: 1; see Table and FIGS. 1A and 1B). In other embodiments, the bacterial asparaginase is Erwinia asparaginase (SEQ ID NO:2; see Table).

In some embodiments, a polypeptide of the disclosure comprises an amino acid sequence that is at least 20% identical to the amino acid sequence of a bacterial asparaginase (e.g., E. coli asparaginase (SEQ ID NO:1) or Erwinia asparaginase (SEQ ID NO:2), or a combination thereof). For example, the sequence identity to the bacterial asparaginase can be at least about: 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99%. In some embodiments, the sequence identity is about: 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99%. In some embodiments, the sequence identity is about: 20-99%, 25-99%, 25-95%, 30-99%, 30-98%, 30-95%, 30-90%, 35-98%, 35-97%, 35-90%, 35-85%, 40-97%, 40-96%, 40-85%, 40-80%, 45-96%, 45-95%, 45-80%, 45-75%, 50-95%, 50-90%, 50-75%, 50-70%, 55-90%, 55-85%, 55-70%, 55-65%, 60-85%, 60-80%, 60-65%, 65-80% or 65-75%. In some embodiments, a polypeptide of the disclosure comprises an amino acid sequence that is at least about 50%, at least about 75% or at least about 90% identical to the amino acid sequence of the bacterial asparaginase (e.g., E. coli asparaginase (SEQ ID NO:1) or Erwinia asparaginase (SEQ ID NO:2), or a combination thereof).

As used herein, the term “sequence identity,” refers to the extent to which two nucleotide sequences, or two amino acid sequences, have the same residues at the same positions when the sequences are aligned to achieve a maximal level of identity, expressed as a percentage. For sequence alignment and comparison, typically one sequence is designated as a reference sequence, to which a test sequences are compared. The sequence identity between reference and test sequences is expressed as the percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide or amino acid residue at 70% of the same positions over the entire length of the reference sequence.

Alignment of sequences for comparison to achieve maximal levels of identity can be readily performed by a person of ordinary skill in the art using an appropriate alignment method or algorithm. In some instances, the alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology).

When using a sequence comparison algorithm, test and reference sequences are input into a computer, subsequent coordinates are designated, if necessary, and sequence algorithm program parameters are designated. The sequence comparison algorithm then calculates the percent sequence identity for the test sequence(s) relative to the reference sequence, based on the designated program parameters. A commonly used tool for determining percent sequence identity is Protein Basic Local Alignment Search Tool (BLASTP) available through National Center for Biotechnology Information, National Library of Medicine, of the United States National Institutes of Health. (Altschul et al., 1990).

In some embodiments, the amino acid sequence of a polypeptide of the disclosure comprises at least one amino acid substitution, relative to the amino acid sequence of a bacterial asparaginase (e.g., E. coli asparaginase (SEQ ID NO:1) or Erwinia asparaginase (SEQ ID NO:2), or a combination thereof). In some embodiments, the amino acid sequence of a polypeptide of the disclosure comprises at least one amino acid substitution, relative to the amino acid sequence of E. coli asparaginase (SEQ ID NO:1).

In some embodiments, the number of amino acid substitutions in a polypeptide of the disclosure relative to the amino acid sequence of a bacterial asparaginase is at least about: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60. In some embodiments, the number of amino acid substitutions is at least about: 5, 6, 10, 15, 20, 25, 30, 35, 40, 45, 50, 55 or 60. In some embodiments, the number of amino acid substitutions is up to about: 60, 55, 50, 45, 40, 35, 30, 25, 20, 15, 10, 6 or 5. In some embodiments, the number of amino acid substitutions is about: 1-60, 1-55, 2-55, 2-50, 3-50, 3-45, 4-45, 4-40, 5-40, 5-35, 6-35, 6-30, 7-30, 7-25, 8-25, 8-20, 9-20, 9-15, 10-15, 5-60, 10-60, 10-55, 15-55, 15-50, 20-50, 20-45, 25-45, 25-40 or 30-40. In some embodiments, the number of amino acid substitutions is about: 10-35, 10-33, 11-33, 11-31, 12-31, 12-29, 13-29, 13-27, 14-27 or 14-25.

The amino acid substitution(s) in a polypeptide of the disclosure can be substitutions with a canonical amino acid or a non-canonical amino acid. Non-canonical amino acids include, but are not limited to D amino acids, such as D versions of the canonical L-amino acids.

In some embodiments, an amino acid substitution is a conservative substitution. The term “conservative amino acid substitution(s)” or “conservative substitution(s)” refers to an amino acid substitution having a value of 0 or greater in BLOSUM62.

In some embodiments, an amino acid substitution is a highly conservative substitution. The term “highly conservative amino acid substitution(s)” or “highly conservative substitution(s)” refers to an amino acid substitution having a value of at least 1 (e.g., at least 2) in BLOSUM62.

In some embodiments, a polypeptide of the disclosure comprises at least one amino acid substitution, relative to the amino acid sequence of E. coli asparaginase (SEQ ID NO:1). In some embodiments, the polypeptide comprises about 5-60 amino acid substitutions, relative to the amino acid sequence of E. coli asparaginase (SEQ ID NO:1). In some embodiments, the amino acid substitutions include at least one conservative substitution. In some embodiments, the amino acid substitutions include at least one highly conservative substitution. In some embodiments, the majority or all amino acid substitutions in the de-immunized asparaginase polypeptide are conservative substitutions. In some embodiments, the majority or all amino acid substitutions in the de-immunized asparaginase polypeptide are highly conservative substitutions.

In some embodiments, a polypeptide of the disclosure comprises an amino acid substitution at a position corresponding to one or more of positions 1, 2, 3, 33, 34, 36, 37, 38, 43, 47, 49, 50, 52, 54, 64, 68, 71, 72, 75, 76, 78, 79, 80, 104, 106, 138, 139, 140, 142, 143, 144, 192, 196, 199, 200, 201, 202, 207, 208, 209, 210, 224, 225, 229, 232, 233, 252, 255, 256, 258, 259, 262, 263, 265, 287, 288, 311, 312, 314, 316, 317, 318, 321, 324 or 325 of SEQ ID NO:1, or a combination thereof. In some embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or all 65 of the positions are substituted. In some embodiments, about: 1-65, 5-65, 5-60, 10-60, 10-55, 15-55, 15-50, 20-50, 20-45, 25-45, 25-40, 30-40 or 30-35 of the positions are substituted.

The amino acid positions identified in this disclosure by reference to a number generally refer to positions of amino acids in SEQ ID NO:1. Positions and amino acid residues in a polypeptide of the disclosure that correspond to particular positions identified in SEQ ID NO:1 can be readily determined by the skilled artisan, for example, by utilizing a multiple sequence alignment (MSA), such as the one shown in FIGS. 1A and 1B.

In some embodiments, the polypeptide comprises an amino acid substitution at a position corresponding to one or more of positions 37, 54, 64, 68, 75, 140, 143, 199, 201, 202, 225, 232, 252, 255, 259, 263, 311, 312, 314, 317, 318, 321, 324 or 325 of SEQ ID NO:1, or a combination thereof. In some embodiments, 2, 3, 4, 5, 6, 7, 8, 9, 10, 11, 12, 13, 14, 15, 16, 17, 18, 19, 20, 21, 22, 23 or all 24 of the positions are substituted. In some embodiments, about: 1-24, 2-24, 2-20, 4-20, 4-16, 5-20, 5-15, 6-16, 6-14, 8-14, 8-12 or 10-12 of the positions are substituted.

In some embodiments, the polypeptide comprises an amino acid substitution at a position corresponding to one or more of positions 54, 64, 68, 75, 143, 199, 201, 202, 232, 263, 311, 312, 317, 318 or 324 of SEQ ID NO:1, or a combination thereof. In some embodiments, 2, 3, 4, 5, 6, 7, 8, 9, 10, 11, 12, 13, 14 or all 15 of the positions are substituted.

In some embodiments, a polypeptide of the disclosure comprises an amino acid substitution at a position corresponding to one or more of positions:
a) 54, 201, 202, 232, 263 or 311 SEQ ID NO:1, or a combination thereof,
b) 2, 37, 43, 54, 64, 68, 75, 139, 140, 143, 196, 199, 202, 209, 210, 225, 252, 255, 259, 263, 312, 314, 317, 318, 321, 324 or 325 SEQ ID NO:1, or a combination thereof,
c) 2, 33, 34, 37, 54, 64, 68, 71, 139, 143, 196, 199, 200, 202, 207, 209, 225, 252, 259, 262, 263, 312, 317, 318, 321, 324 or 325 SEQ ID NO:1, or a combination thereof,
d) 1, 2, 3, 34, 37, 38, 47, 54, 64, 68, 75, 139, 140, 143, 196, 199, 202, 209, 225, 233, 259, 262, 263, 312, 317, 318, 321, 324 or 325 SEQ ID NO:1, or a combination thereof,
e) 1, 2, 37, 49, 54, 64, 68, 71, 75, 139, 140, 143, 196, 199, 200, 202, 207, 209, 210, 225, 252, 255, 259, 262, 263, 312, 317, 318, 321, 324, 325 SEQ ID NO:1, or a combination thereof,
f) 33, 37, 38, 54, 64, 68, 75, 139, 140, 143, 199, 200, 202, 207, 209, 252, 255, 263, 287, 312, 314, 317, 318, 321, 324, 325 SEQ ID NO:1, or a combination thereof,
g) 37, 54, 68, 75, 140, 143, 199, 200, 225, 263, 312 or 314 SEQ ID NO:1, or a combination thereof,
h) 1, 2, 3, 33, 34, 36, 37, 38, 43, 47, 49, 54, 64, 68, 72, 75, 76, 78, 79, 80, 106, 138, 139, 140, 142, 143, 144, 192, 196, 199, 200, 201, 202, 208, 209, 224, 225, 229, 232, 233, 252, 255, 256, 258, 259, 263, 265, 287, 288, 311, 312, 314, 316, 317, 318, 321, 324 or 325 SEQ ID NO:1, or a combination thereof,
i) 37, 50, 54, 64, 68, 75, 140, 196, 199, 200, 202, 209, 225, 252, 255, 259, 262, 263, 287, 312, 314, 317, 318, 321, 324 or 325 SEQ ID NO:1, or a combination thereof, or
j) 1, 2, 3, 33, 34, 37, 38, 43, 47, 49, 50, 52, 54, 64, 68, 71, 75, 78, 104, 106, 139, 143, 199, 207, 209, 210, 225, 233, 252, 255, 259, 263, 288, 312, 317, 318, 321, 324 or 325 SEQ ID NO:1, or a combination thereof.

In some embodiments, the polypeptide comprises an amino acid substitution at a position corresponding to one or more of positions 54, 201, 202, 232, 263 or 311 of SEQ ID NO:1, or a combination thereof. In some embodiments, 2, 3, 4, 5 or all 6 of the positions are substituted.

In some embodiments, the polypeptide comprises an amino acid substitution at a position corresponding to one or more of positions 2, 37, 43, 54, 64, 68, 75, 139, 140, 143, 196, 199, 202, 209, 210, 225, 252, 255, 259, 263, 312, 314, 317, 318, 321, 324 or 325 of SEQ ID NO:1, or a combination thereof. In some embodiments, 2, 3, 4, 5, 6, 7, 8, 9, 10, 11, 12, 13, 14, 15, 16, 17, 18, 19, 20, 21, 22, 23, 24, 25, 26 or all 27 of the positions are substituted.

In some embodiments, the polypeptide comprises an amino acid substitution at a position corresponding to one or more of positions 2, 33, 34, 37, 54, 64, 68, 71, 139, 143, 196, 199, 200, 202, 207, 209, 225, 252, 259, 262, 263, 312, 317, 318, 321, 324 or 325 of SEQ ID NO:1, or a combination thereof. In some embodiments, 2, 3, 4, 5, 6, 7, 8, 9, 10, 11, 12, 13, 14, 15, 16, 17, 18, 19, 20, 21, 22, 23, 24, 25, 26 or all 27 of the positions are substituted.

In some embodiments, the polypeptide comprises an amino acid substitution at a position corresponding to one or more of positions 1, 2, 3, 34, 37, 38, 47, 54, 64, 68, 75, 139, 140, 143, 196, 199, 202, 209, 225, 233, 259, 262, 263, 312, 317, 318, 321, 324 or 325 of SEQ ID NO:1, or a combination thereof. In some embodiments, 2, 3, 4, 5, 6, 7, 8, 9, 10, 11, 12, 13, 14, 15, 16, 17, 18, 19, 20, 21, 22, 23, 24, 25, 26, 27, 28 or all 29 of the positions are substituted.

In some embodiments, the polypeptide comprises an amino acid substitution at a position corresponding to one or more of positions 1, 2, 37, 49, 54, 64, 68, 71, 75, 139, 140, 143, 196, 199, 200, 202, 207, 209, 210, 225, 252, 255, 259, 262, 263, 312, 317, 318, 321, 324 or 325 of SEQ ID NO: 1, or a combination thereof. In some embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30 or all 31 of the positions are substituted.

In some embodiments, the polypeptide comprises an amino acid substitution at a position corresponding to one or more of positions 33, 37, 38, 54, 64, 68, 75, 139, 140, 143, 199, 200, 202, 207, 209, 252, 255, 263, 287, 312, 314, 317, 318, 321, 324 or 325 of SEQ ID NO:1, or a combination thereof. In some embodiments, 2, 3, 4, 5, 6, 7, 8, 9, 10, 11, 12, 13, 14, 15, 16, 17, 18, 19, 20, 21, 22, 23, 24, 25 or all 26 of the positions are substituted.

In some embodiments, the polypeptide comprises an amino acid substitution at a position corresponding to one or more of positions 37, 54, 68, 75, 140, 143, 199, 200, 225, 263, 312 or 314 of SEQ ID NO:1, or a combination thereof. In some embodiments, 2, 3, 4, 5, 6, 7, 8, 9, 10, 11 or all 12 of the positions are substituted.

In some embodiments, the polypeptide comprises an amino acid substitution at a position corresponding to one or more of positions 1, 2, 3, 33, 34, 36, 37, 38, 43, 47, 49, 54, 64, 68, 72, 75, 76, 78, 79, 80, 106, 138, 139, 140, 142, 143, 144, 192, 196, 199, 200, 201, 202, 208, 209, 224, 225, 229, 232, 233, 252, 255, 256, 258, 259, 263, 265, 287, 288, 311, 312, 314, 316, 317, 318, 321, 324 or 325 of SEQ ID NO:1, or a combination thereof. In some embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 or all 58 of the positions are substituted.

In some embodiments, the polypeptide comprises an amino acid substitution at a position corresponding to one or more of positions 37, 50, 54, 64, 68, 75, 140, 196, 199, 200, 202, 209, 225, 252, 255, 259, 262, 263, 287, 312, 314, 317, 318, 321, 324 or 325 of SEQ ID NO:1, or a combination thereof. In some embodiments, 2, 3, 4, 5, 6, 7, 8, 9, 10, 11, 12, 13, 14, 15, 16, 17, 18, 19, 20, 21, 22, 23, 24, 25 or all 26 of the positions are substituted.

In some embodiments, the polypeptide comprises an amino acid substitution at a position corresponding to one or more of positions 1, 2, 3, 33, 34, 37, 38, 43, 47, 49, 50, 52, 54, 64, 68, 71, 75, 78, 104, 106, 139, 143, 199, 207, 209, 210, 225, 233, 252, 255, 259, 263, 288, 312, 317, 318, 321, 324 or 325 of SEQ ID NO:1, or a combination thereof. In some embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or all 39 of the positions are substituted.

In some embodiments, the polypeptide comprises an amino acid substitution at a position corresponding to the position in SEQ ID NO:1 selected from: L1I, L1K or L1M; P2K; N3K; E33D; N34E or N34K; V36I; N37A, N37E or N37K; A38G, A38L or A38R; K43A; N47D or N47E; K49D, K49R or K49T; G50A or G50V; Q52E; V54K, V54M or V54R; N64E; T68E, T68K or T68R; K71E, K71L or K71R; K72R; T75D, T75E or T75K; D76L; D78E; K79Q; T80Y; K104T; D106N or D106S; D138H; K139D, K139E, K139P or K139Q; A140E or A140R; A142K; N143G; R144Q; T192Q; K196L or K196P; S199K, S199L or S199P; D200A, D200E or D200L; T201S; P202E; K207G, K207N or K207S; L208I; N209D, N209E or N209T; E210A or E210S; S224F; D225A, D225S or D225V; K229E; V232I; D233E or D233K; S252E or S252L; D255E, D2551, D255K or D255Q; T256A; A258K; T259E, T259K or T259Q; K262A, K262E or K262R; T263D, T263N or T263R; T265I; A287E; K288A or K288Q; T311I or T311V; Q312K or Q312L; K314N; P316R; Q317A, Q317E or Q317R; Q318A or Q318E; Q321A, Q321E, Q321K or Q321R; N324D or N324R; Q325E, Q325R or Q325T of SEQ ID NO:1, or a combination thereof. In some embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or all 65 of the positions are substituted.

In some embodiments, the polypeptide comprises an amino acid substitution at a position corresponding to the position in SEQ ID NO:1 selected from: L1I, L1K or LIM; P2K; N3K; E33D; N34E or N34K; N37A, N37E or N37K; A38G, A38L or A38R; K43A; N47D or N47E; K49D, K49R or K49T; G50A or G50V; V54K, V54M or V54R; N64E; T68E, T68K or T68R; K71E, K71L or K71R; T75D, T75E or T75K; D78E; D106N or D106S; D138H; K139D, K139E, K139P or K139Q; A140E or A140R; N143G; K196L or K196P; S199K, S199L or S199P; D200A, D200E or D200L; T201S; P202E; K207G, K207N or K207S; N209D, N209E or N209T; E210A or E210S; D225A, D225S or D225V; V232I; D233E or D233K; S252E or S252L; D255E, D255I, D255K or D255Q; T259E, T259K or T259Q; K262A, K262E or K262R; T263D, T263N or T263R; A287E; K288A or K288Q; T311I or T311V; Q312K or Q312L; K314N; Q317A, Q317E or Q317R; Q318A or Q318E; Q321A, Q321E, Q321K or Q321R; N324D or N324R; Q325E, Q325R or Q325T of SEQ ID NO:1, or a combination thereof. In some embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or all 48 of the positions are substituted.

In some embodiments, the polypeptide comprises an amino acid substitution at a position corresponding to the position in SEQ ID NO:1 selected from: N37A, N37E or N37K; V54K or V54R; N64E; T68E, T68K or T68R; T75D, T75E or T75K; A140E or A140R; N143G; S199K, S199L or S199P; T201S; P202E; D225A, D225S or D225V; V232I; S252E or S252L; D255E, D2551, D255K or D255Q; T259E, T259K or T259Q; T263D, T263N or T263R; T311I or T311V; Q312K or Q312L; K314N; Q317A, Q317E or Q317R; Q318A or Q318E; Q321A, Q321E, Q321K or Q321R; N324D or N324R; Q325E, Q325R or Q325T of SEQ ID NO:1, or a combination thereof. In some embodiments, 2, 3, 4, 5, 6, 7, 8, 9, 10, 11, 12, 13, 14, 15, 16, 17, 18, 19, 20, 21, 22, 23 or all 24 of the positions are substituted.

In some embodiments, the polypeptide comprises an amino acid substitution at a position corresponding to the position in SEQ ID NO:1 selected from: V54K, N64E, T68K, T75E, N143G, S199P, T201S, P202E, V232I, T263D, T311I, Q312K, Q317E, Q318E or N324D of SEQ ID NO: 1, or a combination thereof. In some embodiments, the polypeptide comprises at least 1, at least 2, at least 3, at least 4, at least 5, at least 6, at least 7, at least 8, at least 9, at least 10, at least 11, at least 12, at least 13, at least 14 or all 15 substitutions at a position corresponding to the position in SEQ ID NO:1 selected from: V54K, N64E, T68K, T75E, N143G, S199P, T201S, P202E, V232I, T263D, T311I, Q312K, Q317E, Q318E or N324D of SEQ ID NO: 1, or a combination thereof.

In some embodiments, the polypeptide comprises an amino acid substitution at a position corresponding to the position in SEQ ID NO:1 selected from:
a) V54K, T201S, P202E, V232I, T263D or T311I, or a combination thereof,
b) P2K, N37K, K43A, V54K, N64E, T68K, T75D, K139Q, A140R, N143G, K196P, S199P, P202E, N209T, E210S, D225V, S252E, D255K, T259E, T263N, Q312K, K314N, Q317E, Q318E, Q321R, N324D or Q325T, or a combination thereof,
c) P2K, E33D, N34K, N37E, V54K, N64E, T68K, K71L, K139P, N143G, K196P, S199L, D200A, P202E, K207S, N209T, D225V, S252E, T259K, K262E, T263D, Q312K, Q317E, Q318E, Q321K, N324D or Q325T, or a combination thereof,
d) L1I, P2K, N3K, N34E, N37E, A38R, N47E, V54M, N64E, T68K, T75E, K139Q, A140R, N143G, K196P, S199P, P202E, N209T, D225S, D233E, T259K, K262E, T263D, Q312K, Q317E, Q318E, Q321R, N324D or Q325T, or a combination thereof,
e) L1I, P2K, N37K, K49R, V54K, N64E, T68K, K71E, T75K, K139Q, A140R, N143G, K196L, S199P, D200L, P202E, K207S, N209D, E210A, D225A, S252E, D255Q, T259Q, K262A, T263D, Q312K, Q317E, Q318E, Q321E, N324D or Q325R, or a combination thereof;
f) E33D, N37A, A38L, V54K, N64E, T68K, T75D, K139E, A140R, N143G, S199P, D200L, P202E, K207N, N209T, S252L, D2551, T263N, A287E, Q312K, K314N, Q317R, Q318E, Q321E, N324D or Q325T, or a combination thereof,
g) N37K, V54K, T68R, T75D, A140R, N143G, S199P, D200L, D225S, T263N, Q312K or K314N, or a combination thereof,
h) L1K, P2K, N3K, E33D, N34E, V36I, N37A, A38R, K43A, N47E, K49D, V54M, N64E, T68K, K72R, T75E, D76L, D78E, K79Q, T80Y, D106S, D138H, K139P, A140E, A142K, N143G, R144Q, T192Q, K196P, S199L, D200A, T201S, P202E, L208I, N209E, S224F, D225V, K229E, V232I, D233K, S252E, D255K, T256A, A258K, T259E, T263N, T265I, A287E, K288Q, T311V, Q312K, K314N, P316R, Q317R, Q318E, Q321R, N324D or Q325T, or a combination thereof,
i) N37A, G50V, V54K, N64E, T68K, T75D, A140R, K196L, S199L, D200E, P202E, N209D, D225V, S252E, D255K, T259E, K262R, T263R, A287E, Q312K, K314N, Q317R, Q318E, Q321E, N324R or Q325R, or a combination thereof; or
j) LIM, P2K, N3K, E33D, N34E, N37A, A38G, K43A, N47D, K49T, G50A, Q52E, V54R, N64E, T68E, K71R, T75E, D78E, K104T, D106N, K139D, N143G, S199K, K207G, N209T, E210S, D225S, D233E, S252E, D255E, T259E, T263N, K288A, Q312L, Q317A, Q318A, Q321A, N324D or Q325E, or a combination thereof.

In some embodiments, the polypeptide comprises an amino acid substitution at a position corresponding to the position in SEQ ID NO:1 selected from: V54K, T201S, P202E, V232I, T263D or T311I, or a combination thereof. In some embodiments, 2, 3, 4, 5 or all 6 of the positions are substituted.

In some embodiments, the polypeptide comprises an amino acid substitution at a position corresponding to the position in SEQ ID NO:1 selected from: P2K, N37K, K43A, V54K, N64E, T68K, T75D, K139Q, A140R, N143G, K196P, S199P, P202E, N209T, E210S, D225V, S252E, D255K, T259E, T263N, Q312K, K314N, Q317E, Q318E, Q321R, N324D or Q325T, or a combination thereof. In some embodiments, 2, 3, 4, 5, 6, 7, 8, 9, 10, 11, 12, 13, 14, 15, 16, 17, 18, 19, 20, 21, 22, 23, 24, 25, 26 or all 27 of the positions are substituted.

In some embodiments, the polypeptide comprises an amino acid substitution at a position corresponding to the position in SEQ ID NO:1 selected from: P2K, E33D, N34K, N37E, V54K, N64E, T68K, K71L, K139P, N143G, K196P, S199L, D200A, P202E, K207S, N209T, D225V, S252E, T259K, K262E, T263D, Q312K, Q317E, Q318E, Q321K, N324D or Q325T, or a combination thereof. In some embodiments, 2, 3, 4, 5, 6, 7, 8, 9, 10, 11, 12, 13, 14, 15, 16, 17, 18, 19, 20, 21, 22, 23, 24, 25, 26 or all 27 of the positions are substituted.

In some embodiments, the polypeptide comprises an amino acid substitution at a position corresponding to the position in SEQ ID NO:1 selected from: L1I, P2K, N3K, N34E, N37E, A38R, N47E, V54M, N64E, T68K, T75E, K139Q, A140R, N143G, K196P, S199P, P202E, N209T, D225S, D233E, T259K, K262E, T263D, Q312K, Q317E, Q318E, Q321R, N324D or Q325T, or a combination thereof. In some embodiments, 2, 3, 4, 5, 6, 7, 8, 9, 10, 11, 12, 13, 14, 15, 16, 17, 18, 19, 20, 21, 22, 23, 24, 25, 26, 27, 28 or all 29 of the positions are substituted.

In some embodiments, the polypeptide comprises an amino acid substitution at a position corresponding to the position in SEQ ID NO:1 selected from: L1I, P2K, N37K, K49R, V54K, N64E, T68K, K71E, T75K, K139Q, A140R, N143G, K196L, S199P, D200L, P202E, K207S, N209D, E210A, D225A, S252E, D255Q, T259Q, K262A, T263D, Q312K, Q317E, Q318E, Q321E, N324D or Q325R, or a combination thereof. In some embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30 or all 31 of the positions are substituted.

In some embodiments, the polypeptide comprises an amino acid substitution at a position corresponding to the position in SEQ ID NO:1 selected from: E33D, N37A, A38L, V54K, N64E, T68K, T75D, K139E, A140R, N143G, S199P, D200L, P202E, K207N, N209T, S252L, D255I, T263N, A287E, Q312K, K314N, Q317R, Q318E, Q321E, N324D or Q325T, or a combination thereof. In some embodiments, 2, 3, 4, 5, 6, 7, 8, 9, 10, 11, 12, 13, 14, 15, 16, 17, 18, 19, 20, 21, 22, 23, 24, 25 or all 26 of the positions are substituted.

In some embodiments, the polypeptide comprises an amino acid substitution at a position corresponding to the position in SEQ ID NO:1 selected from: N37K, V54K, T68R, T75D, A140R, N143G, S199P, D200L, D225S, T263N, Q312K or K314N, or a combination thereof. In some embodiments, 2, 3, 4, 5, 6, 7, 8, 9, 10, 11 or all 12 of the positions are substituted.

In some embodiments, the polypeptide comprises an amino acid substitution at a position corresponding to the position in SEQ ID NO:1 selected from: L1K, P2K, N3K, E33D, N34E, V36I, N37A, A38R, K43A, N47E, K49D, V54M, N64E, T68K, K72R, T75E, D76L, D78E, K79Q, T80Y, D106S, D138H, K139P, A140E, A142K, N143G, R144Q, T192Q, K196P, S199L, D200A, T201S, P202E, L208I, N209E, S224F, D225V, K229E, V232I, D233K, S252E, D255K, T256A, A258K, T259E, T263N, T265I, A287E, K288Q, T311V, Q312K, K314N, P316R, Q317R, Q318E, Q321R, N324D or Q325T, or a combination thereof. In some embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 or all 58 of the positions are substituted.

In some embodiments, the polypeptide comprises an amino acid substitution at a position corresponding to the position in SEQ ID NO:1 selected from: N37A, G50V, V54K, N64E, T68K, T75D, A140R, K196L, S199L, D200E, P202E, N209D, D225V, S252E, D255K, T259E, K262R, T263R, A287E, Q312K, K314N, Q317R, Q318E, Q321E, N324R or Q325R, or a combination thereof. In some embodiments, 2, 3, 4, 5, 6, 7, 8, 9, 10, 11, 12, 13, 14, 15, 16, 17, 18, 19, 20, 21, 22, 23, 24, 25, 26 or all 27 of the positions are substituted.

In some embodiments, the polypeptide comprises an amino acid substitution at a position corresponding to the position in SEQ ID NO:1 selected from: L1M, P2K, N3K, E33D, N34E, N37A, A38G, K43A, N47D, K49T, G50A, Q52E, V54R, N64E, T68E, K71R, T75E, D78E, K104T, D106N, K139D, N143G, S199K, K207G, N209T, E210S, D225S, D233E, S252E, D255E, T259E, T263N, K288A, Q312L, Q317A, Q318A, Q321A, N324D or Q325E, or a combination thereof. In some embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or all 39 of the positions are substituted.

In still other embodiments, a polypeptide of the disclosure comprises a conservative substitution, a highly conservative substitution or no substitution, at the positions corresponding to the interfacial residues, active site residues or a combination thereof. The active sites are positions corresponding to positions 12, 25, 89, 90, 162 and 283 of SEQ ID NO:1 (FIGS. 1A and 1B). The interfacial residues are positions corresponding to positions 23, 41, 60, 63, 94, 121, 122, 123, 151, 156, 163, 164, 165, 167, 175, 176, 181, 184, 186, 218, 222, 248, 250, 251, 272, 275, 277, 281 and 300 of SEQ ID NO:1.

In some embodiments, a polypeptide of the disclosure comprises a conservative substitution, a highly conservative substitution or no substitution, at the positions corresponding to each of positions 12, 25, 89, 90, 162 and 283 of SEQ ID NO:1. In some embodiments, the polypeptide does not comprise a substitution at the positions corresponding to each of positions 12, 25, 89, 90, 162 and 283 of SEQ ID NO:1.

In some embodiments, a polypeptide of the disclosure comprises a conservative substitution, a highly conservative substitution or no substitution, at the positions corresponding to each of positions 23, 41, 60, 63, 94, 121, 122, 123, 151, 156, 163, 164, 165, 167, 175, 176, 181, 184, 186, 218, 222, 248, 250, 251, 272, 275, 277, 281 and 300 of SEQ ID NO:1. In some embodiments, the polypeptide does not comprise a substitution at the positions corresponding to each of positions 23, 41, 60, 63, 94, 121, 122, 123, 151, 156, 163, 164, 165, 167, 175, 176, 181, 184, 186, 218, 222, 248, 250, 251, 272, 275, 277, 281 and 300 of SEQ ID NO:1.

In some embodiments, the polypeptide comprises a conservative substitution, a highly conservative substitution or no substitution, at the positions corresponding to each of positions 12, 23, 25, 41, 60, 63, 89, 90, 94, 121, 122, 123, 151, 156, 162, 163, 164, 165, 167, 175, 176, 181, 184, 186, 218, 222, 248, 250, 251, 272, 275, 277, 281, 283 and 300 of SEQ ID NO:1. In some embodiments, the polypeptide does not comprise a substitution at the positions corresponding to each of positions 12, 23, 25, 41, 60, 63, 89, 90, 94, 121, 122, 123, 151, 156, 162, 163, 164, 165, 167, 175, 176, 181, 184, 186, 218, 222, 248, 250, 251, 272, 275, 277, 281, 283 and 300 of SEQ ID NO:1.

In some embodiments, the polypeptide does not comprise a T→V or T→Q substitution at the position corresponding to position 192 of SEQ ID NO:1. In some embodiments, the polypeptide does not comprise a substitution at the position corresponding to position 192 of SEQ ID NO:1.

In some embodiments, the polypeptide does not comprise a substitution of the T residue at the position corresponding to position 80 of SEQ ID NO:1 with an aromatic amino acid. In some embodiments, the polypeptide does not comprise a substitution at the position corresponding to position 80 of SEQ ID NO:1.

The de-immunized asparaginase polypeptides disclosed herein, also referred to as resurfaced bacterial asparaginases, include full-length asparaginases (e.g., variants of full-length wild type bacterial asparaginases), functional fragments of wild type bacterial asparaginases, and variants of functional fragments of wild type bacterial asparaginases.

In some embodiments, the polypeptide comprises a truncation of 1-50 amino acids corresponding to the amino acids at positions 1-50 of E. coli asparaginase (SEQ ID NO:1). For example, the N-terminal truncation can be about: 1, 2, 3, 4, 5, 6, 7, 8, 9, 10, 11, 12, 13, 14, 15, 16, 17, 18, 19, 20, 25, 30, 35, 40, 45, or 50 amino acids; or 1-45, 1-20, 2-45, 2-40, 2-20, 2-19, 3-40, 3-35, 3-19, 3-18, 4-35, 4-30, 4-18, 4-17, 5-30, 5-25, 5-17, 5-16, 6-25, 6-20, 6-16, 6-15, 7-20, 7-19, 7-15, 7-14, 8-19, 8-18, 8-14, 8-13, 9-18, 9-17, 9-13, 9-12, 10-17, 10-16, 10-12, 11-16, 11-15, 12-15 or 12-14 amino acids. In some embodiments, the polypeptide comprises a truncation of 1-20 amino acids corresponding to the amino acids at positions 1-20 of E. coli asparaginase (SEQ ID NO: 1).

In some embodiments, the polypeptide comprises a truncation of 1-50 amino acids corresponding to the amino acids at positions 276-326 of E. coli asparaginase (SEQ ID NO: 1). For example, the C-terminal truncation can be about: 1, 2, 3, 4, 5, 6, 7, 8, 9, 10, 11, 12, 13, 14, 15, 16, 17, 18, 19, 20, 25, 30, 35, 40, 45, or 50 amino acids; or 1-45, 1-20, 2-45, 2-40, 2-20, 2-19, 3-40, 3-35, 3-19, 3-18, 4-35, 4-30, 4-18, 4-17, 5-30, 5-25, 5-17, 5-16, 6-25, 6-20, 6-16, 6-15, 7-20, 7-19, 7-15, 7-14, 8-19, 8-18, 8-14, 8-13, 9-18, 9-17, 9-13, 9-12, 10-17, 10-16, 10-12, 11-16, 11-15, 12-15 or 12-14 amino acids. In some embodiments, the polypeptide comprises a truncation of 1-20 amino acids corresponding to the amino acids at positions 306-326 of E. coli asparaginase (SEQ ID NO:1).

In some embodiments, a polypeptide of the disclosure comprises one or more amino acid deletions at a position other than the N- or C-terminus, relative to the amino acid sequence of a bacterial asparaginase (e.g., E. coli asparaginase (SEQ ID NO:1) or Erwinia asparaginase (SEQ ID NO:2), or a combination thereof). In some embodiments, the deletion is about 1-50 amino acids, for example, about: 1, 2, 3, 4, 5, 6, 7, 8, 9, 10, 11, 12, 13, 14, 15, 16, 17, 18, 19, 20, 25, 30, 35, 40, 45, or 50 amino acids; or 1-45, 1-20, 2-45, 2-40, 2-20, 2-19, 3-40, 3-35, 3-19, 3-18, 4-35, 4-30, 4-18, 4-17, 5-30, 5-25, 5-17, 5-16, 6-25, 6-20, 6-16, 6-15, 7-20, 7-19, 7-15, 7-14, 8-19, 8-18, 8-14, 8-13, 9-18, 9-17, 9-13, 9-12, 10-17, 10-16, 10-12, 11-16, 11-15, 12-15 or 12-14 amino acids.

In some embodiments, a polypeptide of the disclosure comprises one or more amino acid insertions, relative to the amino acid sequence of a bacterial asparaginase (e.g., E. coli asparaginase (SEQ ID NO:1) or Erwinia asparaginase (SEQ ID NO:2), or a combination thereof). In some embodiments, the insertion is about 1-50 amino acids, for example, about: 1, 2, 3, 4, 5, 6, 7, 8, 9, 10, 11, 12, 13, 14, 15, 16, 17, 18, 19, 20, 25, 30, 35, 40, 45, or 50 amino acids; or 1-45, 1-20, 2-45, 2-40, 2-20, 2-19, 3-40, 3-35, 3-19, 3-18, 4-35, 4-30, 4-18, 4-17, 5-30, 5-25, 5-17, 5-16, 6-25, 6-20, 6-16, 6-15, 7-20, 7-19, 7-15, 7-14, 8-19, 8-18, 8-14, 8-13, 9-18, 9-17, 9-13, 9-12, 10-17, 10-16, 10-12, 11-16, 11-15, 12-15 or 12-14 amino acids.

In some embodiments, a polypeptide of the disclosure comprises the amino acid sequence of SEQ ID NO:3.

SEQ ID NO: 3
X₁X₂X₃ITILATGGTIAGGGDSATKSNYTVGKVGVX₄X₅LX₆X₇
X₈VPQLX₉DIAX₁₀VX₁₁X₁₂EX₁₃VX₁₄NIGSQDMNDX₁₅VWLX₁₆
LAX₁₇X₁₈INX₁₉X₂₀CX₂₁X₂₂X₂₃DGFVITHGTDTMEETAYFLD
LTVX₂₄CX₂₅KPVVMVGAMRPSTSMSADGPFNLYNAVVTAAX₂₆
X₂₇X₂₈SX₂₉X₃₀X₃₁GVLVVMNDTVLDGRDVTKTNTTDVATFK
SVNYGPLGYIHNGKIDYQRX₃₂PARX₃₃HTX₃₄X₃₅X₃₆X₃₇FD
VSX₃₈X₃₉X₄₀X₄₁LPKVGIVYNYANAX₄₂X₄₃LPAX₄₄ALX₄₅X₄₆
AGYDGIVSAGVGNGNLYKX₄₇VFX₄₈X₄₉LX₅₀X₅₁AAX₅₂X₅₃
GX₅₄AVVRSSRVPTGATTQDAEVDDX₅₅X₅₆YGFVASGTLNP
QKARVLLQLALX₅₇X₅₈TX₅₉DX₆₀X₆₁X₆₂IQX₆₃IFX₆₄X₆₅Y

In some embodiments,
1) X₁ is not L;
2) X₂ is not P;
3) X₃ is not N;
4) X₄ is not E;
5) X₅ is not N;
6) X₆ is not V;
7) X₇ is not N;
8) X₈ is not A;
9) X₉ is not K;
10) X₁₀ is not N;
11) X₁₁ is not K;
12) X₁₂ is not G;
13) X₁₃ is not Q;
14) X₁₄ is not V;
15) X₁₅ is not N;
16) X₁₆ is not T;
17) X₁₇ is not K;
18) X₁₈ is not K;
19) X₁₉ is not T;
20) X₂₀ is not D;
21) X₂₁ is not D;
22) X₂₂ is not K;
23) X₂₃ is not T;
24) X₂₄ is not K;
25) X₂₅ is not D;
26) X₂₆ is not D;
27) X₂₇ is not K;
28) X₂₈ is not A;
29) X₂₉ is not A;
30) X₃₀ is not N;
31) X₃₁ is not R;
32) X₃₂ is not T;
33) X₃₃ is not K;
34) X₃₄ is not S;
35) X₃₅ is not D;
36) X₃₆ is not T;
37) X₃₇ is not P;
38) X₃₈ is not K;
39) X₃₉ is not L;
40) X₄₀ is not N;
41) X₄₁ is not E;
42) X₄₂ is not S;
43) X₄₃ is not D;
44) X₄₄ is not K;
45) X₄₅ is not V;
46) X₄₆ is not D;
47) X₄₇ is not S;
48) X₄₈ is not D;
49) X₄₉ is not T;
50) X₅₀ is not A;
51) X₅₁ is not T;
52) X₅₂ is not K;
53) X₅₃ is not T;
54) X₅₄ is not T;
55) X₅₅ is not A;
56) X₅₆ is not K;
57) X₅₇ is not T;
58) X₅₈ is not Q;
59) X₅₉ is not K;
60) X₆₀ is not P;
61) X₆₁ is not Q;
62) X₆₂ is not Q;
63) X₆₃ is not Q;
64) X₆₄ is not N R; or
65) X₆₅ is not Q,
or a combination thereof.

In some embodiments,
1) X₁ is L, I, K or M;
2) X₂ is P or K;
3) X₃ is N or K;
4) X₄ is E or D;
5) X₅ is N, E or K;
6) X₆ is V or I;
7) X₇ is N, A, E or K;
8) X₈ is A, G, L or R;
9) X₉ is K or A;
10) X₁₀ is N, D or E;
11) X₁₁ is K, D, R or T;
12) X₁₂ is G, A or V;
13) X₁₃ is Q or E;
14) X₁₄ is V, K, M or R;
15) X₁₅ is N or E;
16) X₁₆ is T, E, K or R;
17) X₁₇ is K, E, L or R;
18) X₁₈ is K or R;
19) X₁₉ is T, D, E or K;
20) X₂₀ is D or L;
21) X₂₁ is D or E;
22) X₂₂ is K or Q;
23) X₂₃ is T or Y;
24) X₂₄ is K or T;
25) X₂₅ is D, N or S;
26) X₂₆ is D or H;
27) X₂₇ is K, D, E, P or Q;
28) X₂₈ is A, E or R;
29) X₂₉ is A or K;
30) X₃₀ is N or G;
31) X₃₁ is R or Q;
32) X₃₂ is T or Q;
33) X₃₃ is K, L or P;
34) X₃₄ is S, K, L or P;
35) X₃₅ is D, A, E or L;
36) X₃₆ is T or S;
37) X₃₇ is P or E;
38) X₃₈ is K, G, N or S;
39) X₃₉ is L or I;
40) X₄₀ is N, D, E or T;
41) X₄₁ is E, A or S;
42) X₄₂ is S or F;
43) X₄₃ is D, A, S or V;
44) X₄₄ is K or E;
45) X₄₅ is V or I;
46) X₄₆ is D, E or K;
47) X₄₇ is S, E or L;
48) X₄₈ is D, E, I, K or Q;
49) X₄₉ is T or A;
50) X₅₀ is A or K;
51) X₅₁ is T, E, K or Q;
52) X₅₂ is K, A, E or R;
53) X₅₃ is T, D, N or R;
54) X₅₄ is T or I;
55) X₅₅ is A or E;
56) X₅₆ is K, A or Q;
57) X₅₇ is T, I or V;
58) X₅₈ is Q, K or L;
59) X₅₉ is K or N;
60) X₆₀ is P or R;
61) X₆₁ is Q, A, E or R;
62) X₆₂ is Q, A or E;
63) X₆₃ is Q, A, E, K or R;
64) X₆₄ is N, D or R; or
65) X₆₅ is Q, E, R or T,
or a combination thereof.

In some embodiments,
1) X₁ is I, K or M;
2) X₂ is K;
3) X₃ is K;
4) X₄ is D;
5) X₅ is E or K;
6) X₆ is I;
7) X₇ is A, E or K;
8) X₈ is G, L or R;
9) X₉ is A;
10) X₁₀ is D or E;
11) X₁₁ is D, R or T;
12) X₁₂ is A or V;
13) X₁₃ is E;
14) X₁₄ is K, M or R;
15) X₁₅ is E;
16) X₁₆ is E, K or R;
17) X₁₇ is E, L or R;
18) X₁₈ is R;
19) X₁₉ is D, E or K;
20) X₂₀ is L;
21) X₂₁ is E;
22) X₂₂ is Q;
23) X₂₃ is Y;
24) X₂₄ is T;
25) X₂₅ is N or S;
26) X₂₆ is H;
27) X₂₇ is D, E, P or Q;
28) X₂₈ is E or R;
29) X₂₉ is K;
30) X₃₀ is G;
31) X₃₁ is Q;
32) X₃₂ is Q;
33) X₃₃ is L or P;
34) X₃₄ is K, L or P;
35) X₃₅ is A, E or L;
36) X₃₆ is S;
37) X₃₇ is E;
38) X₃₈ is G, N or S;
39) X₃₉ is I;
40) X₄₀ is D, E or T;
41) X₄₁ is A or S;
42) X₄₂ is F;
43) X₄₃ is A, S or V;
44) X₄₄ is E;
45) X₄₅ is I;
46) X₄₆ is E or K;
47) X₄₇ is E or L;
48) X₄₈ is E, I, K or Q;
49) X₄₉ is A;
50) X₅₀ is K;
51) X₅₁ is E, K or Q;
52) X₅₂ is A, E or R;
53) X₅₃ is D, N or R;
54) X₅₄ is I;
55) X₅₅ is E;
56) X₅₆ is A or Q;
57) X₅₇ is I or V;
58) X₅₈ is K or L;
59) X₅₉ is N;
60) X₆₀ is R;
61) X₆₁ is A, E or R;
62) X₆₂ is A or E;
63) X₆₃ is A, E, K or R;
64) X₆₄ is D or R; or
65) X₆₅ is E, R or T,
or a combination thereof.

In some embodiments, the polypeptide comprises an amino acid sequence set forth in any one of SEQ ID NOs:4-13, or a variant thereof (e.g., a variant comprising an amino acid sequence having at least about 50% identity to the amino acid sequence of set forth in any one of SEQ ID NOs:4-13). In some embodiments, the polypeptide comprises an amino acid sequence set forth in any one of SEQ ID NOs:4-13 (see Table and FIGS. 1A and 1B). In some embodiments, a polypeptide of the disclosure comprises an amino acid sequence set forth in any one of SEQ ID NO:8, SEQ ID NO:9, SEQ ID NO:11 or SEQ ID NO:13.

In some embodiments, a polypeptide of the disclosure comprises an amino acid sequence that is at least 45% identical to the amino acid sequence of any one of SEQ ID NOs:4-13, or a combination thereof. In some embodiments, the sequence identity is at least about: 50%, 55%, 60%, 65%, 70%, 75%, 80%, 85%, 90%, 91%, 92%, 93%, 94%, 95%, 96%, 97%, 98% or 99%. In some embodiments, the sequence identity is at least about: 70%, 80%, 90% or 95%. In some embodiments, the sequence identity is about: 50%, 55%, 60%, 65%, 70%, 75%, 80%, 85%, 90%, 91%, 92%, 93%, 94%, 95%, 96%, 97%, 98% or 99%. In some embodiments, the sequence identity is about: 50-99%, 50-98%, 50-95%, 55-98%, 55-97%, 55-95%, 55-90%, 60-97%, 60-96%, 60-90%, 60-85%, 65-96%, 65-95%, 65-80%, 65-75%, 70-95%, 70-90%, 75-90%, 75-85% or 80-85%.

In some embodiments, a polypeptide of the disclosure comprises at least one amino acid substitution, relative to the amino acid sequence of any one of SEQ ID NOs:4-13, or a combination thereof. In some embodiments, the polypeptide comprises about 1-60 amino acid substitutions, relative to the amino acid sequence of any one of SEQ ID NOs:4-13, or a combination thereof, for example, about: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 amino acid substitutions, relative to the amino acid sequence of any one of SEQ ID NOs:4-13, or a combination thereof. In some embodiments, the polypeptide comprises at least about: 1, 2, 3, 4, 5, 6, 7, 8, 9, 10, 11, 12, 13, 14, 15, 16, 17, 18, 19, 20, 25 or 30 amino acid substitutions, relative to the amino acid sequence of any one of SEQ ID NOs:4-13, or a combination thereof. In some embodiments, the polypeptide comprises up to about: 30, 25, 20, 15, 14, 13, 12, 11, 10, 9, 8, 7, 6, 5, 4, 3, 2 amino acid substitutions, relative to the amino acid sequence of any one of SEQ ID Nos:4-13, or a combination thereof. In some embodiments, the polypeptide comprises about: 1-60, 1-55, 1-20, 1-19, 1-15, 2-55, 2-50, 2-19, 2-18, 2-15, 2-14, 3-50, 3-45, 3-18, 3-17, 3-14, 3-13, 4-45, 4-40, 4-17, 4-16, 4-13, 4-12, 5-60, 5-40, 5-35, 5-16, 5-15, 5-12, 5-11, 6-35, 6-30, 6-15, 6-14, 6-11, 6-10, 7-30, 7-25, 7-14, 7-13, 7-10, 7-9, 8-25, 8-20, 8-13, 8-12, 9-20, 9-15, 9-12, 9-11, 10-60, 10-55, 10-15, 15-55, 15-50, 20-50, 20-45, 25-45, 25-40 or 30-40 amino acid substitutions, relative to the amino acid sequence of any one of SEQ ID Nos:4-13, or a combination thereof. In some embodiments, the polypeptide comprises about 5-60 amino acid substitutions, relative to the amino acid sequence of any one of SEQ ID Nos:4-13, or a combination thereof.

In some embodiments, the amino acid substitutions include at least one conservative substitution. In some embodiments, the amino acid substitutions include at least one highly conservative substitution.

In some embodiments, a polypeptide of the disclosure further comprises a leader sequence at the N-terminus. In some embodiments, the leader sequence comprises a sequence set forth in SEQ ID NO:14, SEQ ID NO:15 or SEQ ID NO:16. In some embodiments, the leader sequence comprises a sequence set forth in SEQ ID NO:16. In some embodiments, the leader sequence comprises an amino acid sequence that is at least about 70% identical to the amino acid sequence of SEQ ID NO:14, SEQ ID NO:15 or SEQ ID NO:16, or a combination thereof. In some embodiments, the leader sequence comprises an amino acid sequence that is at least about 70% identical to the amino acid sequence of SEQ ID NO: 16. For example, the sequence identity is at least about: 75%, 80%, 85%, 90%, 91%, 92%, 93%, 94%, 95%, 96%, 97%, 98% or 99%. In some embodiments, the sequence identity is about: 75%, 80%, 85%, 90%, 91%, 92%, 93%, 94%, 95%, 96%, 97%, 98% or 99%. In some embodiments, the sequence identity is about: 75-99%, 75-98%, 80-98%, 80-97%, 85-97%, 85-96%, 90-96%, 90-95%, 91-95%, 91-94% or 92-94%.

In some embodiments, the leader sequence comprises at least one amino acid substitution, relative to the amino acid sequence set forth in SEQ ID NO:14, SEQ ID NO:15 or SEQ ID NO: 16, or a combination thereof. The polypeptide of claim 4, wherein the polypeptide comprises at least about 1-5 amino acid substitutions (e.g., about 1, 2, 3, 4, or 5 amino acid substitutions), relative to the amino acid sequence of SEQ ID NO:14, SEQ ID NO: 15 or SEQ ID NO:16, or a combination thereof.

In some embodiments, the polypeptide further comprises a peptide tag. In some embodiments, the peptide tag comprises a poly-histidine sequence, for example, 2-10 consecutive histidine amino acids, e.g., a 2×His tag, 3×His tag, 4×His tag, 5×His tag, 6×His, 7×His tag, 8×His tag, 9×His tag, or 10×His tag. In some embodiments, the peptide tag comprises, consists of, or consists essentially of a 6×His tag.

In some embodiments, the polypeptide further comprises a TEV protease cleavage site. In some embodiments, the TEV protease cleavage site is set forth in SEQ ID NO:18.

In some embodiments, the polypeptide is conjugated to a heterologous moiety. The term “conjugated” refers to attached, via a covalent or noncovalent interaction. Conjugation can employ any of suitable linking agents. Non-limiting examples include peptide linkers, compound linkers, and chemical cross-linking agents.

In some embodiments, the heterologous moiety is a therapeutic agent, a diagnostic agent or a combination thereof. In some embodiments, the heterologous moiety is polyethylene glycol (PEG), hexadecanoic acid, hydrogels, nanoparticles, multimerization domains and carrier peptides.

In some embodiments, the nanoparticle is a lipid nanoparticle. In some embodiments, the nanoparticle is a polymer nanoparticle. In some embodiments, the polymer is an amphiphilic polymer. In other embodiments, the polymer is a hydrophobic or hydrophilic polymer. Non-limiting examples of polymers include poly(lactic acid)-poly(ethylene glycol), poly(lactic-co-glycolic acid)-poly(ethylene glycol), poly(lactic-co-glycolic) acid (PLGA), poly(lactic-co-glycolic acid)-d-α-tocopheryl polyethylene glycol succinate, poly(lactic-co-glycolic acid)-ethylene oxide fumarate, poly(glycolic acid)-poly(ethylene glycol), polycaprolactone-poly(ethylene glycol), or any salts thereof. In some embodiments, the polymer nanoparticle comprises poly(lactic-co-glycolic) acid (PLGA).

In some embodiments, the carrier polypeptide is albumin or an Fc polypeptide.

In some embodiments, a polypeptide of the disclosure:
a) can be produced with an overall yield of purified asparaginase of at least about 1 mg asparaginase per liter of E. coli culture;
b) can be produced with an enzyme yield of at least about 50%, relative to E. coli asparaginase (SEQ ID NO:1);
c) binds an anti-asparaginase antibody with an affinity that is at least 10% lower, relative to the E. coli asparaginase (SEQ ID NO: 1),
d) or any combination thereof.

In some embodiments, a polypeptide of the disclosure can be produced with an overall yield of purified asparaginase of at least about 1 mg asparaginase per liter of E. coli culture. In some embodiments, a polypeptide of the disclosure can be produced with an overall yield of purified asparaginase of at least about: 10, 20, 50, 100, 150, 200, 250, 300, 350, 400, 450, 500, 600, 700, 800, 900 or 1,000 mg asparaginase per liter of E. coli culture. In some embodiments, a polypeptide of the disclosure can be produced with an overall yield of purified asparaginase of about 1-1,000, 1-800, 1-700, 1-600, 1-500, 10-1,000, 10-900, 20-900, 20-800, 50-800, 50-700, 100-700, 100-600, 150-600, 150-500, 200-500, 200-400, 250-400, 250-350 or 300-350 mg asparaginase per liter of E. coli culture. In some embodiments, a polypeptide of the disclosure can be produced with an overall yield of purified asparaginase of about 1-500 mg asparaginase per liter of E. coli culture.

In some embodiments, a polypeptide of the disclosure can be produced with an enzyme yield of at least about 10% relative to E. coli asparaginase (SEQ ID NO: 1), for example, at least about: 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99%, relative to E. coli asparaginase (SEQ ID NO: 1). In some embodiments, a polypeptide of the disclosure can be produced with an enzyme yield of about: 10-99%, 15-99%, 15-95%, 20-95%, 20-90%, 25-90%, 25-85%, 30-85%, 30-80%, 35-80%, 35-75%, 40-75%, 40-70%, 45-70%, 45-65%, 50-65% or 50-60%, relative to E. coli asparaginase (SEQ ID NO:1). In some embodiments, the polypeptide can be produced with an enzyme yield of at least about 50% relative to E. coli asparaginase (SEQ ID NO: 1),

In some embodiments, a polypeptide of the disclosure binds an anti-asparaginase antibody with an affinity that is at least about 1% lower, relative to the E. coli asparaginase (SEQ ID NO: 1), for example, at least about: 2%, 3%, 4%, 5%, 6%, 7%, 8%, 9%, 10%, 15%, 20%, 25%, 30%, 35%, 40%, 45%, 50%, 55%, 60%, 65%, 70%7, 75%, 80%, 85%, 90% or 95% lower, relative to the E. coli asparaginase (SEQ ID NO: 1). In some embodiments, the polypeptide binds an anti-asparaginase antibody with an affinity that is about: 1-99%, 2-99%, 2-95%, 3-95%, 3-90%, 4-90%, 4-85%, 5-85%, 5-80%, 6-80%, 6-75%, 7-75%, 7-70%, 8-70%, 8-65%, 9-65%, 9-60%, 10-60%, 10-55%, 15-55%, 15-50%, 20-50%, 20-45%, 25-45%, 25-40% or 30-40% lower, relative to the E. coli asparaginase (SEQ ID NO: 1). In some embodiments, a polypeptide of the disclosure binds an anti-asparaginase antibody with an affinity that is at least about 10% lower, relative to the E. coli asparaginase (SEQ ID NO: 1).

### Fusion Proteins

In another aspect, the disclosure provides a fusion protein comprising one or more of the polypeptides described herein.

The term “fusion protein” refers to a synthetic, semi-synthetic or recombinant single protein molecule. A fusion protein can comprise all or a portion of two or more different proteins and/or polypeptides that are attached by covalent bonds (e.g., peptide bonds).

Fusion proteins of the disclosure can be produced recombinantly or synthetically, using routine methods and reagents that are well known in the art. For example, a fusion protein of the disclosure can be produced recombinantly in a suitable host cell (e.g., bacteria) according to methods known in the art. See, e.g., Current Protocols in Molecular Biology, Second Edition, Ausubel et al. eds., John Wiley & Sons, 1992; and Molecular Cloning: a Laboratory Manual, 2nd edition, Sambrook et al., 1989, Cold Spring Harbor Laboratory Press. For example, a nucleic acid molecule comprising a nucleotide sequence encoding a fusion protein described herein can be introduced and expressed in suitable host cell (e.g., E. coli), and the expressed fusion protein can be isolated/purified from the host cell (e.g., in inclusion bodies) using routine methods and readily available reagents. For example, DNA fragments coding for different protein sequences (e.g., a light-responsive domain, a heterologous peptide component) can be ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers that give rise to complementary overhangs between two consecutive nucleic acid fragments that can subsequently be annealed and re-amplified to generate a chimeric nucleic acid sequence (see Ausubel et al., Current Protocols in Molecular Biology, 1992).

### Polynucleotides, Expression Vectors, Expression Host Cells

In another aspect, the disclosure provides one or more polynucleotides encoding any one of the polypeptides or fusion proteins described herein. In some embodiments, the polypeptide or fusion protein of the disclosure is encoded by a single polynucleotide. In some embodiments, the polypeptide or fusion protein of the disclosure is encoded by multiple polynucleotides.

In some embodiments, the polynucleotide comprises a nucleotide sequence that is codon-optimized for a chosen host cell.

In another aspect, the disclosure provides an expression vector comprising any one or more of the polynucleotides described herein.

The term “expression vector” refers to a replicable nucleic acid from which one or more proteins can be expressed when the expression vector is transformed into a suitable expression host cell.

In some embodiments, the expression vector further comprises an expression control polynucleotide sequence operably linked to the polynucleotide, a polynucleotide sequence encoding a selectable marker, or both. In some embodiments, the expression control polynucleotide sequence comprises a promoter sequence, an enhancer sequence, or both. In some embodiments, the expression control polynucleotide sequence comprises an inducible promoter sequence. The term “promoter” refers to a region of DNA to which RNA polymerase binds and initiates the transcription of a gene. The term “operably linked” means that the nucleic acid is positioned in the recombinant polynucleotide, e.g., vector, in such a way that enables expression of the nucleic acid under control of the element (e.g., promoter) to which it is linked. The term “selectable marker element” is an element that confers a trait suitable for artificial selection. Selectable marker elements can be negative or positive selection markers.

In another aspect, the disclosure provides an expression host cell comprising any one or more of the polynucleotides or expression vectors described herein.

The term “expression host cell” refers to a cell useful for receiving, maintaining, reproducing and amplifying a vector.

Non-limiting examples of expression host cells include bacterial cells such as E. coli strains BL21, CU1783, DH5a, gram positive bacteria (which may be capable of better secretion), yeast such as Pichia, as well as mammalian cells such as Chinese hamster ovary (CHO) cells and Human embryonic kidney (HEK) 293 cells, etc.

### Compositions

In another aspect, the disclosure provides a composition comprising any one of the polypeptides or fusion proteins described herein. In some embodiments, the composition is a pharmaceutical composition.

In some embodiments, the composition (e.g., pharmaceutical composition) further comprises pharmaceutically acceptable carriers, excipients, stabilizers, diluents or tonifiers (Remington's Pharmaceutical Sciences 16th edition, Osol, A. Ed. (1980)). Suitable pharmaceutically acceptable carriers, excipients, or stabilizers are nontoxic to recipients at the dosages and concentrations employed. Non-limiting examples of pharmaceutically acceptable carriers, excipients, stabilizers, diluents or tonifiers include buffers (e.g., phosphate, citrate, histidine), antioxidants (e.g., ascorbic acid or methionine), preservatives, proteins (e.g., serum albumin, gelatin or immunoglobulins); hydrophilic polymers, amino acids, carbohydrates (e.g., monosaccharides, disaccharides, glucose, mannose or dextrins); chelating agents (e.g., EDTA), sugars (e.g., sucrose, mannitol, trehalose or sorbitol), salt-forming counter-ions (e.g., sodium), metal complexes (e.g., Zn-protein complexes); non-ionic surfactants (e.g., Tween), PLURONICS™ and polyethylene glycol (PEG).

In some embodiments, the composition (e.g., pharmaceutical composition) of the disclosure is formulated for a suitable administration schedule and route. Non-limiting examples of administration routes include oral, rectal, mucosal, intravenous, intramuscular, subcutaneous and topical, etc. In some embodiments, the composition (e.g., pharmaceutical composition) of the disclosure is stored in the form of an aqueous solution or a dried formulation (e.g., lyophilized).

In some embodiments, the composition is formulated to be administered by infusion (e.g., intravenous infusion).

In some embodiments, the composition is formulated to be administered with a second therapeutic agent as a combination therapy.

### Methods of Use

In another aspect, the disclosure provides a method of reducing a level of asparagine or asparagine-containing product in a biological fluid, comprising contacting the biological fluid with an effective amount of any one of the polypeptides, fusion proteins, compositions or pharmaceutical composition described herein.

In some embodiments, the biological fluid is from a mammalian subject. In some embodiments, the biological fluid is used in food production (e.g., the method reduces acrylamide build up in frying starchy foods).

In another aspect, the disclosure provides a method of treating a mammalian subject in need thereof, comprising administering an effective amount of any one of the polypeptides, fusion proteins, compositions or pharmaceutical composition described herein, to the mammalian subject.

In another aspect, the disclosure provides a method of treating cancer in a mammalian subject in need thereof, comprising administering an effective amount of any one of the polypeptides, fusion proteins, compositions or pharmaceutical composition described herein, to the mammalian subject.

In some embodiments, the pharmaceutical composition comprising a pharmaceutically acceptable carrier and, an active ingredient, wherein the active ingredient comprises any one of the polypeptides or fusion proteins described herein.

The term “subject” or “patient” refers to a mammal (e.g., a human). In some embodiments, the subject is a mammal. In some embodiments, the subject is a mammal selected from the group consisting of a dog, a cat, a mouse, a rat, a hamster, a guinea pig, a horse, a pig, a sheep, a cow, a chimpanzee, a macaque, a cynomolgus, and a human. In some embodiments, the subject is a primate. In some embodiments, the subject is a human.

In some embodiments, the human subject is an infant (less than 1 year old). In some embodiments, the human subject is less than 11 years old. In some embodiments, the human subject is 11 years or older. In some embodiments, the human subject is 12 years or older. In some embodiments, the human subject is 12-17 years old. In some embodiments, the human subject is less than 18 years old. In some embodiments, the human subject is an adult (18 years or older). In some embodiments, the human subject is 40 years or older, e.g., at least: 45, 50, 55, 60, 65, 70, 75, 80, 85, 90 years old. In some embodiments, the human subject is elderly (65 years or older).

A subject to be treated according to the methods described herein may be one who has been diagnosed with a particular condition, or one at risk of developing such conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

In some embodiments, the mammalian subject has cancer, e.g., an asparagine-sensitive cancer. In some embodiments, the cancer is a hematological cancer. In some embodiments, the hematological cancer is acute lymphoblastic leukemia (ALL), non-Hodgkin's lymphoma or acute myeloid leukemia (AMIL). In some embodiments, the hematological cancer is ALL.

In some embodiments, the mammalian subject has an adverse response to one or more bacterial asparaginases. In some embodiments, the mammalian subject is at risk of developing an adverse response to one or more bacterial asparaginases.

In some embodiments, the adverse response comprises an immune system disorder. In some embodiments, the immune system disorder comprises an immune response (e.g., an allergic reaction) mediated by an antibody that specifically binds at least one bacterial asparaginase. In some embodiments, the bacterial asparaginase comprises E. coli asparaginase (SEQ ID NO: 1), Erwinia asparaginase (SEQ ID NO:2), or a combination thereof. In some embodiments, the bacterial asparaginase is E. coli asparaginase (SEQ ID NO: 1).

In some embodiments, the adverse response comprises serious hypersensitivity reactions (including anaphylaxis), severe pancreatitis and hemorrhagic pancreatitis. Additional non-limiting examples of adverse responses include blood and lymphatic system disorders (e.g., hyperglycemia, decreased white blood count), cardiac disorders (e.g., cardiopulmonary failure), infections, metabolism and nutrition disorders (e.g., hypokalemia) and musculoskeletal and connective tissue disorders (e.g., muscle weakness, parathesia and facial weakness).

In some embodiments, the method described herein reduces the likelihood of the mammalian subject to develop an adverse response to bacterial asparaginase by at least about 10%, e.g., by at least about: 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99%.

In some embodiments, the likelihood of the mammalian subject to develop an adverse response to bacterial asparaginase, using the method described herein, is about 1-90% relative to the likelihood if the subject is administered with a wildtype bacterial asparaginase, for example, about: 2-90%, 2-85%, 3-85%, 3-80%, 4-80%, 4-75%, 5-75%, 5-70%, 6-70%, 6-65%, 7-65%, 7-60%, 8-60%, 8-55%, 9-55%, 9-50%, 10-50%, 10-45%, 15-45%, 15-40%, 20-40%, 20-35%, 25-35% or 25-30%.

“A therapeutically effective amount,” “an effective amount” or “an effective dosage” is an amount effective, at dosages and for periods of time necessary, to achieve a desired therapeutic result (e.g., treatment, healing, inhibition or amelioration of physiological response or condition, etc.). The full therapeutic effect does not necessarily occur by administration of one dose, and may occur only after administration of a series of doses. Thus, a therapeutically effective amount may be administered in one or more administrations. A therapeutically effective amount may vary according to factors such as disease state, age, sex, and weight of a mammal, mode of administration and the ability of a therapeutic, or combination of therapeutics, to elicit a desired response in an individual.

An effective amount of an agent to be administered can be determined by a clinician of ordinary skill using the guidance provided herein and other methods known in the art. Relevant factors include the given agent, the pharmaceutical formulation, the route of administration, the type of disease or disorder, the identity of the subject (e.g., age, sex, weight) or host being treated, and the like. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Determining the dosage for a particular agent, subject and disease is well within the abilities of one of skill in the art. Preferably, the dosage does not cause or produces minimal adverse side effects.

Desired response or desired results include effects at the cellular level, tissue level, or clinical results. As such, “a therapeutically effective amount” or synonym thereto depends upon the context in which it is being applied. For example, in some embodiments it is an amount of the composition sufficient to achieve a treatment response as compared to the response obtained without administration of the composition. In other embodiments, it is an amount that results in a beneficial or desired result in a subject as compared to a control. As defined herein, a therapeutically effective amount of a composition of the present disclosure may be readily determined by one of ordinary skill by routine methods known in the art. Dosage regimen and route of administration may be adjusted to provide the optimum therapeutic response.

In some embodiments, the method is used for prophylactic therapy. In some embodiments, the method is used as first-line therapy. In some embodiments, the method is used as second-line therapy. In some embodiments, the method is used as third-line therapy.

In some embodiments, the method is used for treating cancer. The term “treating” or “treatment” refers to the medical management of a subject with the intent to improve, ameliorate, stabilize (i.e., not worsen), prevent or cure a disease, pathological condition, or disorder-such as the particular indications exemplified herein. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

A therapeutic agent described herein can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the compound and the particular disease to be treated. Administration can be local or systemic as indicated. The preferred mode of administration can vary depending on the particular compound chosen.

In some embodiments, the method further comprises administering a therapeutically effective amount of a second therapeutic agent to the subject.

Administration of the two or more therapeutic agents encompasses co-administration of the therapeutic agents in a substantially simultaneous manner, such as in a pharmaceutical combination. Alternatively, such administration encompasses co-administration in multiple containers, or separate containers (e.g., capsules, powders, and liquids) for each therapeutic agent. Such administration also encompasses use of each type of therapeutic agent in a sequential manner, either at approximately the same time or at different times. The composition described herein and the second therapeutic agent can be administered via the same administration route or via different administration routes.

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

As used herein, the indefinite articles “a,” “an” and “the” should be understood to include plural reference unless the context clearly indicates otherwise.

Throughout this specification and the claims which follow, unless the context requires otherwise, the word “comprise,” and variations such as “comprises” and “comprising”, will be understood to imply the inclusion of, e.g., a stated integer or step or group of integers or steps, but not the exclusion of any other integer or step or group of integer or step. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the disclosure, can in some embodiments, be replaced with the term “consisting of,” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

It should be understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, etcetera.

For all patents, applications, or other reference cited herein, such as non-patent literature and reference sequence information, it should be understood that they are incorporated by reference in their entirety for all purposes as well as for the proposition that is recited. Where any conflict exists between a document incorporated by reference and the present application, this application will control. All information associated with reference gene sequences disclosed in this application, such as GeneIDs or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures), as well as chemical references (e.g., PubChem compound, PubChem substance, or PubChem Bioassay entries, including the annotations therein, such as structures and assays, et cetera), are hereby incorporated by reference in their entirety.

Headings used in this application are for convenience only and do not affect the interpretation of this application.

Preferred features of each of the aspects provided by the disclosure are applicable to all of the other aspects of the disclosure mutatis mutandis and, without limitation, are exemplified by the dependent claims and also encompass combinations and permutations of individual features (e.g., elements, including numerical ranges and exemplary embodiments) of particular embodiments and aspects of the disclosure, including the working examples. For example, particular experimental parameters exemplified in the working examples can be adapted for use in the claimed disclosure piecemeal without departing from the disclosure. For example, for materials that are disclosed, while specific reference of each of the various individual and collective combinations and permutations of these compounds may not be explicitly disclosed, each is specifically contemplated and described herein. Thus, if a class of elements A, B, and C are disclosed as well as a class of elements D, E, and F and an example of a combination of elements A-D is disclosed, then, even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-groups of A-E, B-F, and C-E are specifically contemplated and should be considered disclosed from disclosure of A, B, and C; D, E, and F; and the example combination A-D. This concept applies to all aspects of this application, including elements of a composition of matter and steps of method of making or using the compositions.

The forgoing aspects of the disclosure, as recognized by the person having ordinary skill in the art following the teachings of the specification, can be claimed in any combination or permutation to the extent that they are novel and non-obvious over the prior art-thus, to the extent an element is described in one or more references known to the person having ordinary skill in the art, they may be excluded from the claimed disclosure by, inter alia, a negative proviso or disclaimer of the feature or combination of features.

### EXAMPLES

Neutralizing antibodies (nAbs) or anti-drug antibodies (ADAs) limit the effectiveness of therapeutic proteins, especially those that must be administered over an extended period. This study sought to develop proteins that could evade an existing neutralizing antibody response, without knowing the specific epitope(s) being recognized by nAbs in patients. By drastically modifying the surface residues of a therapeutic asparaginase protein while maintaining its functionality (in a process referred to as “resurfacing”) protein variants that demonstrate reduced binding to ADAs were created. In the following examples, E. coli asparaginase (ASN), a therapeutic which exhibits high rates of ADA development following administration in an oncology setting, was used to demonstrate the ability to generate resurfaced proteins capable of avoiding established ADAs. E. coli ASN, clinically referred to as Elspar®, Asparginase, Oncaspar® or Kidrolase®, is utilized for the treatment of several hematologic malignancies and a front-line therapy for acute lymphoblastic leukemia (ALL). Approximately 30% of ALL patients will development hypersensitivity to E. coli ASN treatment and be placed on Erwinia ASN as a second line therapy.

### Example 1. Computational Design and Sequence Generation of Resurfaced ASNs

Using various structure-based design methodologies and the ASN structure as a template (PDB ID 3eca), surface exposed residues were mutated while maintaining overall predicted structure. ASN exists as a heterotetramer, where the interface of monomers forms the enzymatic core of the protein complex. Two sets of resurfaced ASN designs were created: a “conservative” set where up to 58 surface residues were open for modification that are relatively distant from the enzymatic core and monomer-monomer interfaces, and an “aggressive” set where an additional 27 residues with closer proximity to these regions were allowed to be modified. Fourteen designs were selected from these two sets which represented a mix of conservative (nine designs) and aggressive (five designs), each of which having a mix of the two design methodologies. See Table for protein sequences and FIGS. 1A and 1B for sequence alignment. Overall, the designed sequences exhibited a range of 30-92% sequence identity to the parental protein.

### Example 2. Evaluating Expression and Activity of Resurfaced ASNs

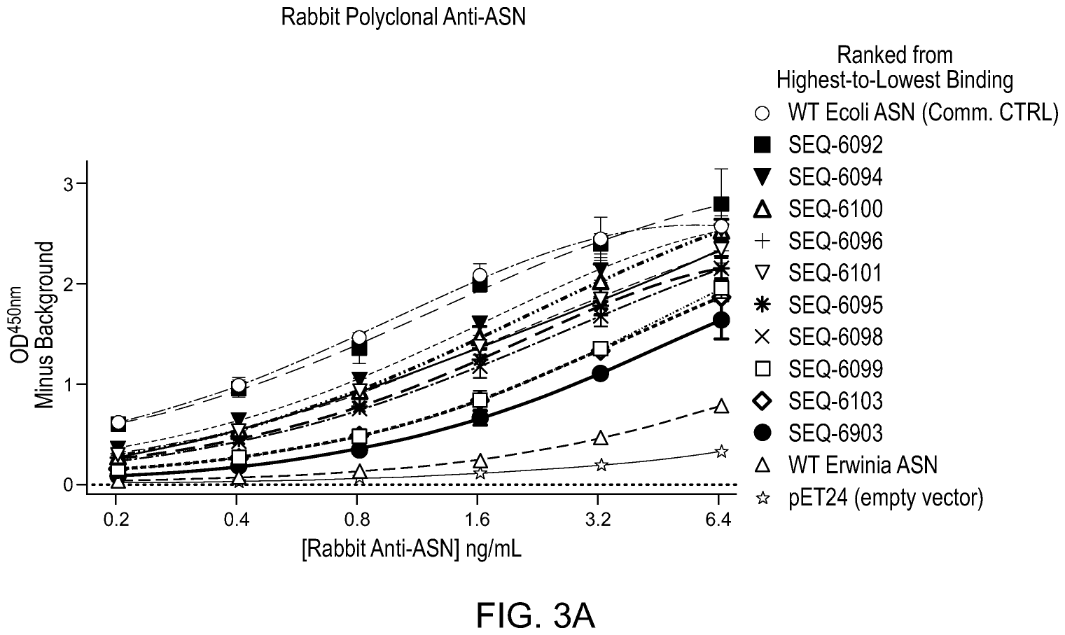

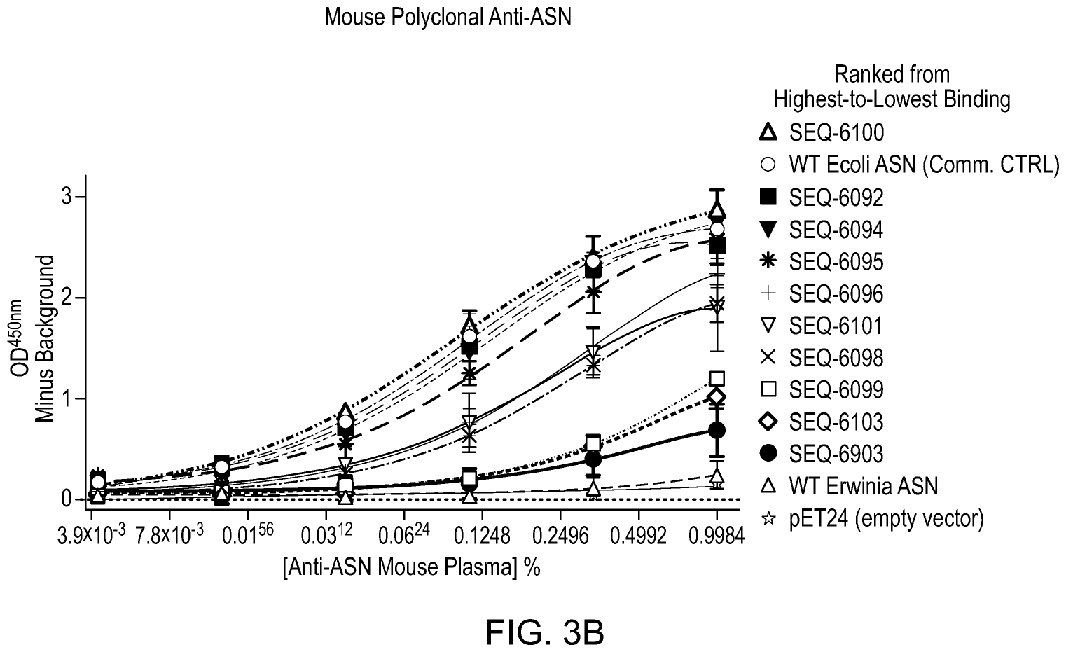

Sequences for the parental and resurfaced ASNs were synthesized, cloned to contain a multi-histidine tag on the N-terminus for purification, and expressed in E. coli at small scale to evaluate their relative expression and activity. In brief, expression vectors were transformed into E. coli in 96-well plates, cells were placed in selection media and grown at 37° C. overnight. The following day a starter culture was transferred to 1 mL of autoinduction media (Sigma Aldrich, St. Louis, MO, Cat. #71300) with antibiotic selection in 96-deep well plates and incubated while shaking overnight at 30° C. The following morning, cells were pelleted and lysed using bug buster lysis media (Sigma Aldrich, St. Louis, MO, Cat. #70584). Lysed cell supernatants were harvested and subjected to expression and activity analysis. Expression was measured using a His Tag ELISA detection kit following the manufacturer's protocols (Genscript Biotech Corporation, Piscataway, NJ, Cat. #L00436). ASN activity was measured by first loading bacterial supernatants onto anti-His tag antibody coated 96-well plates. This allowed for the capture of the artificially expressed ASNs while removing the ASN endogenously expressed by the host E. coli. ASN captured wells were then subjected to an ASN activity colorimetric assay following the manufacturer's instructions (BioVision, Inc., Milpitas, CA, Cat. #K754). See FIGS. 3A and 3B for expression and activity results.

The conservative designs that expressed and exhibited activity contained between 12 and 31 mutations from parental with many of the mutations on the N- and C-terminal regions containing charged residues. These charged residues may contribute to additional salt bridge formation that stabilize the tetrameric complex. Only one of the nine designs that utilized the conservative set of mutations, exhibited activity close to or below background. Most of the individual mutations in that mutant were found in active designs, but it has the lowest sequence identity to the parental protein overall. Thus, it is likely that said sequence contained too many cumulative disruptions to its structure which resulted in destabilization and loss of function.

Deimmunized asparaginase Variant 1 (V1), from the aggressive variant set, exhibited comparable activity to the wild-type (WT) ASN parental protein, and only differs by 6 mutations. Many of the individual mutations found in V1 are as found in other variant designs, which indicates these are positions which are broadly tolerant to modification. Like V1, many of the mutations are shared among the conservative designs—for example V54K, N64E, T68K, T75E, N143G, S199P, P202E, Q312K, Q317E, Q318E, and N324D are not only utilized in nearly every conservative design but also in many aggressive designs. However, in general, the other aggressive designs exhibited activity below parental, likely due to the modification made to residues at the interfacial surface of monomers which may have disrupted tetramer formation or stability. For example, mutating position T192 to either valine or glutamine, occurred in four weakly active aggressive variants, may disrupt a necessary polar interaction at the monomer-monomer interface. In three variants, T80 is mutated to a large bulky aromatic residue, which likely results in a local rearrangement to accommodate the new bulky side chain.

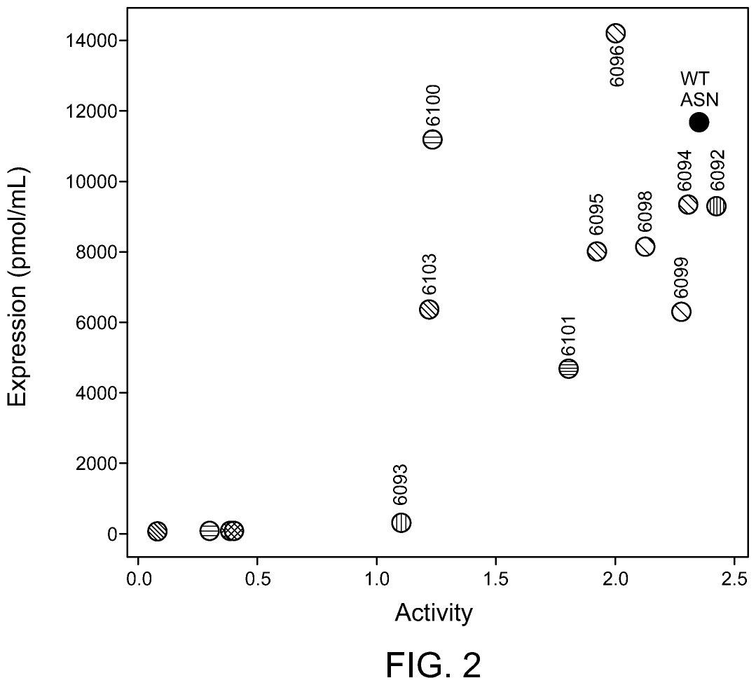

In summary, ten resurfaced ASNs exhibited expression and activity above background levels (as indicated in FIG. 2) and were selected to progress to large-scale production and evaluation for binding to anti-ASN ADAs from mouse, rabbit and human serum.

### Example 3. Evaluating Relative Binding of Resurfaced ASNs to Anti-ASN ADAs

Large-scale expression was performed on the 10 resurfaced ASNs, the parental WT ASN and Erwinia ASN (as a negative control). As described above, E. coli transformed with His-tagged protein expression vectors were grown and induced to express at a 100-mL scale. Bacteria were pelleted and lysed using bug buster lysis media. Lysed supernatants were harvested and bound to Nickel-NTA resin (Thermo Fisher Scientific, Inc. Waltham, MA, Cat. #88221) and target proteins were purified following the manufacturer's batch mode protocol. Purified proteins were quantified by BCA assay (Thermo Fisher Scientific, Inc. Waltham, MA, Cat. #23225) and normalized to the same concentration for downstream assays.

Next, proteins were assayed for their relative binding to anti-ASN ADAs via an Enzyme Linked Immunosorbent Assay (ELISA). Mouse anti-ASN ADAs were generated by repeat intraperitoneal injection of Balbc mice with 15 ug ASN formulated in PBS at a 1:1 ratio with Alum adjuvant (Thermo Fisher Scientific, Inc. Waltham, MA, Cat. #77161). At 17 days post injection initiation and 3 days post the second injection, serum was harvest from the blood of mice and pooled. Rabbit anti-ASN ADAs were sourced commercially (Rockland Immunochemicals, Inc., Limerick, PA, Cat. #100-4171). ASN test articles were resuspended at 5 ug/mL in sodium bicarbonate buffer (BioLegend, Inc., San Diego, CA, Cat. #421701) and coated onto half-area, high-binding 96-well plates (Thermo Fisher Scientific, Inc. Waltham, MA, Cat. #07-200-37) overnight at 4° C. Wells were blocked using blocking buffer (BioLegend, Inc., San Diego, CA, Cat. #421203) for 1 hour at 37° C. Anti-ASN ADAs were serially diluted and used to stain wells containing the ASN test articles, incubating for 1 hour at 37° C. Unbound antibodies were rinsed from wells and either anti-mouse IgG-HRP (Abcam, Cambridge, UK, Cat. #ab97023) or anti-rabbit IgG (Abcam, Cambridge, UK, Cat. #ab6721) were utilized to detect bound ADAs by staining at a 1:5,000 or 1:10,000 dilution, respectively, for 1 hour at 37° C. Finally, the presence of the anti-mouse- and anti-rabbit-HRP was detected by addition of TMB (Thermo Fisher Scientific, Inc. Waltham, MA, Cat. #N301) followed by quenching of the reaction using ELISA Stop solution (Thermo Fisher Scientific, Inc. Waltham, MA, Cat. #N600). Pooled normal mouse serum and polyclonal rabbit isotype controls were used to establish background binding for all test articles. Commercially sourced ASN (Abcam, Cambridge, UK, Cat. #ab73439) stained with anti-ASN ADAs at a fixed dilution were utilized as a positive control. Protein isolated from mock transduced E. coli (pET24 (empty vector)) as described above and Erwinia ASN were used as an additional negative control for binding to anti-ASN ADAs. The Erwinia sequence is as an important control as this molecule is a second line therapy to replace E. coli ASN when patients develop hypersensitivity, due to Erwinia ASN's ability to evade pre-existing anti-E. coli ASN ADAs. Optical density at 450 nm was read out as a measure of relative binding of anti-ASN ADAs to each test article at each dilution.

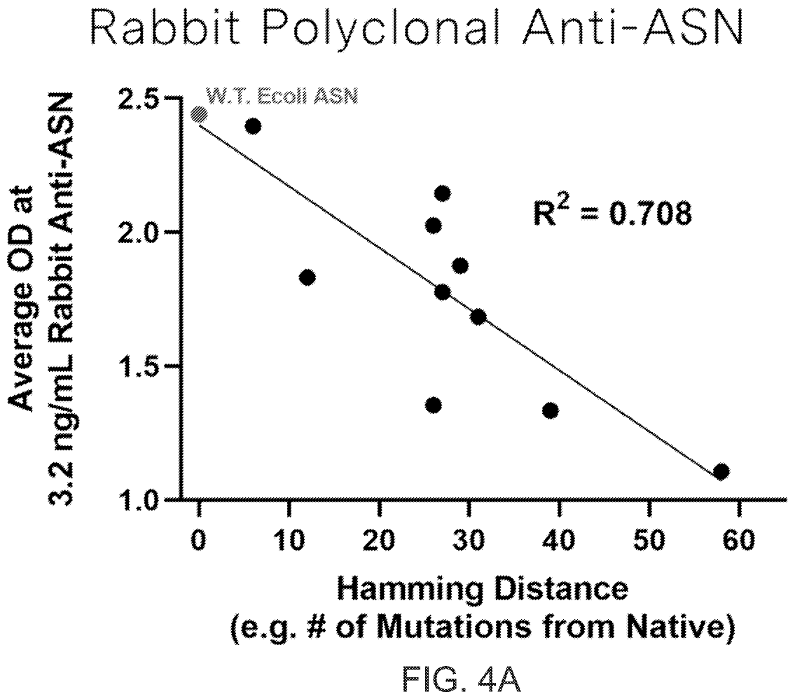

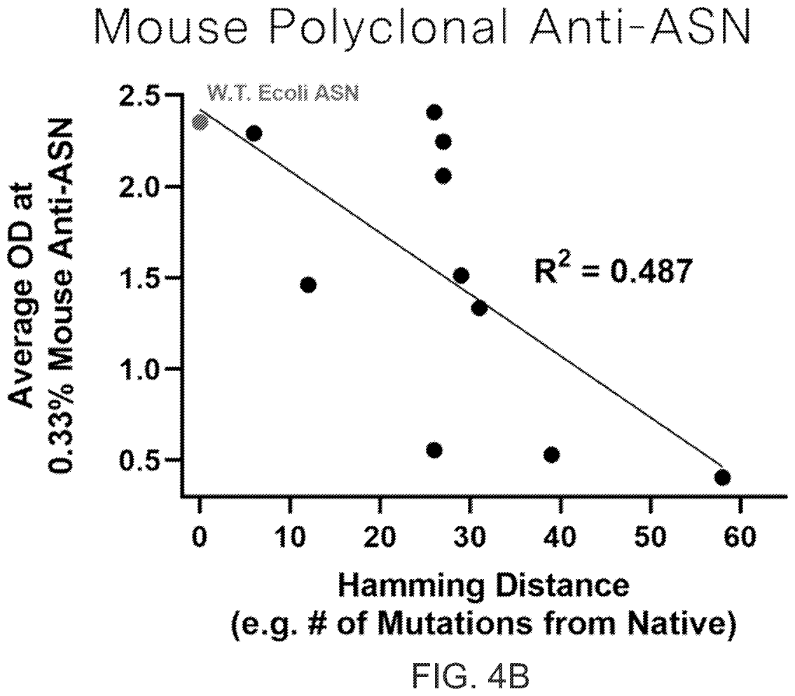

FIGS. 3A and 3B show raw OD^{450 nm} readings and relative binding rankings for each test article. Mock transduced and Erwinia ASN test articles exhibited the lowest binding for both species across all dilutions tested, while E. coli ASN exhibited the highest, or one of the highest, levels of binding compared to all other test articles. Resurfaced ASN variants exhibited a wide range of binding against ADAs from both species, however the relative rankings remained consistent between both. For example, deimmunized asparaginase variants V8, V10, V6 and V5 consistently exhibited the lowest binding to anti-ASN ADAs from both mouse and rabbit. As discussed above, some of the resurfaced variants harbored more mutations with respect to parental E. coli than others, and the most divergent variant had 58 mutations (V8). FIGS. 4A and 4B show the relative binding of ADAs at the dilution which showed the most differentiation in binding signal between the resurfaced ASNs tested (3.2 ng/mL rabbit anti-ASN and 0.33% mouse anti-ASN, respectively). This is compared to the Hamming Distance (or number of mutations away from the parental or native E. coli ASN). For both rabbit and mouse ADAs, an inverse correlation was observed between relative binding of ADAs and Hamming distance, with an R² value of 0.708 for rabbit and 0.487 for mouse ADAs, respectively.

### Example 4. Evaluating Resurfaced ASN Binding to Human Anti-ASN ADAs

A selection of resurfaced ASN candidates representing high, medium, and low binders and their associated controls were produced and purified as described above. These include: E. coli ASN, Erwinia ASN, SEQs 6093, 6094, 6095, 6096, 6099, 6100 and 6103. Activity and relative binding to rabbit and mouse ADAs were confirmed as described above and in human serum.

Clinically, E. coli ASN is utilized as a first-line therapy for the treatment of several hematological malignancies, including Acute Lymphoblastic Leukemia (ALL). A high proportion of ALL patients receiving ASN injections will develop hypersensitivity to the treatment. Serum samples from ALL patients with noted reactions to ASN treatment were sourced from a biobank. Patient samples were assayed for anti-ASN ADAs by subjecting them to the anti-ASN ELISA based assay outlined above. Relative binding of patient-derived ADAs was quantified via ADA titer, or the lowest serum sample concentration at which a positive ADA signal was observed above an established background derived from healthy volunteer serum samples. Anti-human-IgG-HRP (Promega Corporation, Madison, WI, Cat. #W4031) was utilized to detect bound human ADAs from serum or plasma samples. Normal healthy donor serum/plasma was utilized as a negative control and to establish background signal. ALL patient samples exhibiting signal at least two standard deviations above the mean OD^{450 nm} obtained from health donor controls were considered positive for ADAs.

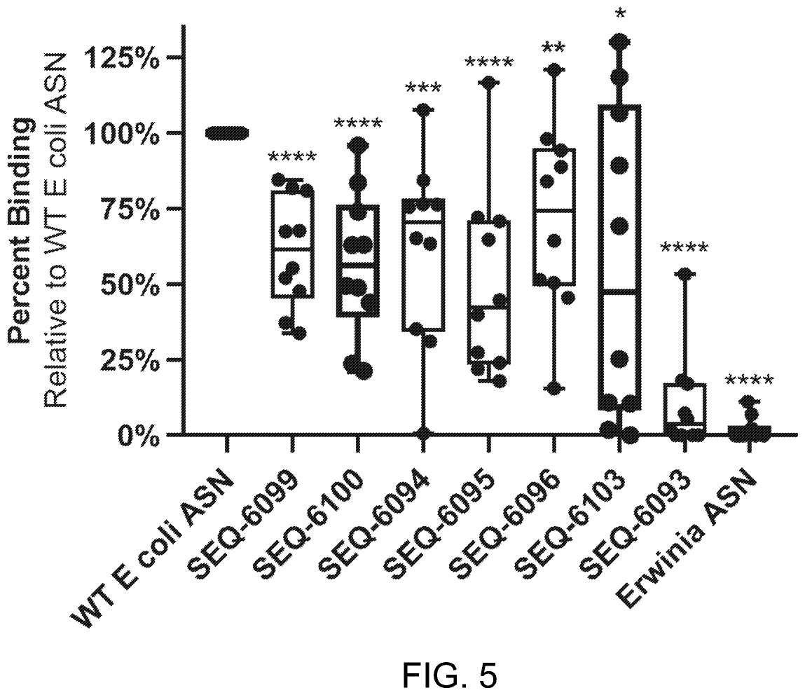

Ten ADA positive samples from independent donors were utilized to assess binding of anti-ASN antibodies to resurfaced ASNs as described above. The resurfaced ASN test articles were assayed for relative binding to human anti-ASN ADAs, with parental E. coli ASN and Erwinia ASN used as controls. The ADA binding signal was normalized within each donor to the signal detected against WT E. coli ASN and Erwinia control, providing a relative measure of ADA binding to resurfaced ASNs. FIG. 5 shows the normalized binding of ADAs against resurfaced ASNs as a percentage binding. All resurfaced ASNs exhibited a statistically significant reduction in their average or mean binding to anti-ASN ADAs from human patient samples relative to E. coli ASN.

The ability to resurface a protein while maintaining that protein's function can afford flexibility in generating substitution (or follow-on) therapies capable of evading pre-existing neutralizing responses or adverse events, without the aid of evolution. These results in three independent species, including humans, support this approach and suggests that a priori the mutational load necessary to escape pre-existing immunity to a therapeutic protein may be predicted.

### Example 5. Reduced Neutralization of Enzymatic Activity from Pre-Existing ADAs by Resurfacing ASNs

One mechanism by which ADAs impact the efficacy of a therapeutic protein is via neutralization of its activity. In the case of ASN, ADAs can neutralize or reduce the ability of the enzyme to convert asparagine to aspartic acid and ammonia. By reducing binding by anti-ASN ADAs, resurfaced ASNs could abrogate their neutralizing effects on enzymatic activity. To verify this, enzymatic assays are performed in the presence or absence of mouse, rabbit or human derived anti-ASN ADAs. WT E. coli ASN and resurfaced ASNs are incubated with ADAs, then the relative levels of enzymatic neutralization are measured via a fluorescent ASN enzymatic assay (Sigma Aldrich, St. Louis, MO, Cat. #MAK007-1KT). Reductions in neutralization of enzymatic activity for resurfaced ASNs compared to WT E. coli ASN following exposure to ADAs are expected, consistent with a model where reductions in ADA binding could result in improvements in potency for resurfaced ASN treatment in patients previously exposed to WT E. coli ASN standard of care treatments.

### Example 6. Reduced Hypersensitivity in Mice Sensitized to WT E. coli ASN by Resurfacing ASNs

A significant proportion of patients treated with WT E. coli ASN will exhibit hypersensitivity responses and will be forced to discontinue such treatments. In this scenario, a replacement ASN capable of avoiding hypersensitivity responses would be needed, such as the current second line therapy Erwinia ASN (or Erwinaze). To evaluate whether the reduced binding of ADAs exhibited by resurfaced ASNs provided by the disclosure leads to reductions in hypersensitivity responses in vivo, the tolerability of resurfaced ASN treatment in mice sensitized to WT E. coli ASN is evaluated. Balbc mice are first given three doses of 15 μg of WT E. coli ASN, one dose per week over the course of three weeks, via tail vein injection. Previous studies have shown that mice begin exhibiting clinical signs of hypersensitivity to WT E. coli ASN treatment starting on the fourth weekly dose. One weeks 4, 5 and 6, mice will receive 15 pg doses of either WT E. coli ASN, Erwinia ASN control or resurfaced ASNs provided by the disclosure. Mice are observed for rapid onset of hypersensitivity symptoms and measured on a 5-point scale of severity (0, no signs of shock; 1, mild shock including itching, ruffling of fur, dyspnea, and decrease in spontaneous activity; 2, moderate shock including prostration, sluggish gait, and slightly activity after prodding; 3, severe shock including complete paresis, no activity following prodding with or without convulsion; 4, death within 30 min). As additional control groups, mice receiving vehicle only injections for the first 3 weeks will then receive doses of WT E. coli ASN, Erwinia ASN or resurfaced ASNs provided by the disclosure for weeks 4, 5 and 6. It is expected that mice sensitized to WT E. coli ASN will exhibit reduced signs of hypersensitivity upon subsequent injections with resurfaced ASNs compared to continued treatment with WT E. coli ASN, consistent with the principle that the reduced binding by ADAs exhibited by resurfaced ASNs also leads to reduced hypersensitivity in vivo. These findings support the use of resurfaced ASNs provided by the disclosure as second line therapies for patients previously exposed and sensitized to E. coli ASN.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

| | | |
|---|---|---|
| | SEQ | |
| Name | ID | Amino Acid Sequence |

Wildtype (WT) Asparaginase

| | | |
|---|---|---|
| WT | No: 1 | LPNITILATGGTIAGGGDSATKSNYTVGKVGVE |
| E. coli | | NLVNAVPQLKDIANVKGEQVVNIGSQDMNDNVW |
| ASN | | LTLAKKINTDCDKTDGFVITHGTDTMEETAYFL |
| (AL-08) | | DLTVKCDKPVVMVGAMRPSTSMSADGPFNLYNA |
| | | VVTAADKASANRGVLVVMNDTVLDGRDVTKTNT |
| | | TDVATFKSVNYGPLGYIHNGKIDYQRTPARKHT |
| | | SDTPFDVSKLNELPKVGIVYNYANASDLPAKAL |
| | | VDAGYDGIVSAGVGNGNLYKSVFDTLATAAKTG |
| | | TAVVRSSRVPTGATTQDAEVDDAKYGFVASGTL |
| | | NPQKARVLLQLALTQTKDPQQIQQIFNQY |
| WT | No: 2 | ADKLPNIVILATGGTIAGSAATGTQTTGYKAGA |
| Erwinia | | LGVDTLINAVPEVKKLANVKGEQFSNMASENMT |
| ASN | | GDVVLKLSQRVNELLARDDVDGVVITHGTDTVE |
| | | ESAYFLHLTVKSDKPVVFVAAMRPATAISADGP |
| | | MNLLEAVRVAGDKQSRGRGVMVVLNDRIGSARY |
| | | ITKTNASTLDTFKANEEGYLGVIIGNRIYYQNR |
| | | IDKLHTTRSVFDVRGLTSLPKVDILYGYQDDPE |
| | | YLYDAAIQHGVKGIVYAGMGAGSVSVRGIAGMR |
| | | KAMEKGVVVIRSTRTGNGIVPPDEELPGLVSDS |
| | | LNPAHARILLMLALTRTSDPKVIQEYFHTY |

Resurfaced Asparaginase

| | | |
|---|---|---|
| Consensus | No: 3 | X₁X₂X₃ITILATGGTIAGGGDSATKSNYTVGKV |
| | | GVX₄X₅LX₆X₇X₈VPQLX₉DIAX₁₀VX₁₁X₁₂E |
| | | X₁₃VX₁₄NIGSQDMNDX₁₅VWLX₁₆LAX₁₇X₁₈ |
| | | INX₁₉X₂₀CX₂₁X₂₂X₂₃DGFVITHGTDTMEET |
| | | AYFLDLTVX₂₄CX₂₅KPVVMVGAMRPSTSMSAD |
| | | GPFNLYNAVVTAAX₂₆X₂₇X₂₈SX₂₉X₃₀X₃₁GVL |
| | | VVMNDTVLDGRDVTKTNTTDVATFKSVNYGP |
| | | LGYIHNGKIDYQRX₃₂PARX₃₃HTX₃₄X₃₅X₃₆ |
| | | X₃₇FDVSX₃₈X₃₉X₄₀X₄₁LPKVGIVYNYANAX₄₂ |
| | | X₄₃LPAX₄₄ALX₄₅X₄₆AGYDGIVSAGVGNG |
| | | NLYKX₄₇VFX₄₈X₄₉LX₅₀X₅₁AAX₅₂X₅₃GX₅₄ |
| | | AVVRSSRVPTGATTQDAEVDDX₅₅X₅₆YGFVA |
| | | SGTLNPQKARVLLQLALX₅₇X₅₈TX₅₉DX₆₀X₆₁ |
| | | X₆₂IQX₆₃IFX₆₄X₆₅Y |
| V1 (SEQ- | No: 4 | LPNITILATGGTIAGGGDSATKSNYTVGKVGVE |
| 6092) | | NLVNAVPQLKDIANVKGEQVKNIGSQDMNDNVW |
| | | LTLAKKINTDCDKTDGFVITHGTDTMEETAYFL |
| | | DLTVKCDKPVVMVGAMRPSTSMSADGPFNLYNA |
| | | VVTAADKASANRGVLVVMNDTVLDGRDVTKTNT |
| | | TDVATFKSVNYGPLGYIHNGKIDYQRTPARKHT |
| | | SDSEFDVSKLNELPKVGIVYNYANASDLPAKAL |
| | | IDAGYDGIVSAGVGNGNLYKSVFDTLATAAKDG |
| | | TAVVRSSRVPTGATTQDAEVDDAKYGFVASGTL |
| | | NPQKARVLLQLALIQTKDPQQIQQIFNQY |
| V2 (SEQ- | No: 5 | LKNITILATGGTIAGGGDSATKSNYTVGKVGVE |
| 6094) | | NLVKAVPQLADIANVKGEQVKNIGSQDMNDEVW |
| | | LKLAKKINDDCDKTDGFVITHGTDTMEETAYFL |
| | | DLTVKCDKPVVMVGAMRPSTSMSADGPFNLYNA |
| | | VVTAADQRSAGRGVLVVMNDTVLDGRDVTKTNT |
| | | TDVATFKSVNYGPLGYIHNGKIDYQRTPARPHT |
| | | PDTEFDVSKLTSLPKVGIVYNYANASVLPAKAL |
| | | VDAGYDGIVSAGVGNGNLYKEVFKTLAEAAKNG |
| | | TAVVRSSRVPTGATTQDAEVDDAKYGFVASGTL |
| | | NPQKARVLLQLALTKTNDPEEIQRIFDTY |
| V3 (SEQ- | No: 6 | LKNITILATGGTIAGGGDSATKSNYTVGKVGVD |
| 6095) | | KLVEAVPQLKDIANVKGEQVKNIGSQDMNDEVW |
| | | LKLALKINTDCDKTDGFVITHGTDTMEETAYFL |
| | | DLTVKCDKPVVMVGAMRPSTSMSADGPFNLYNA |
| | | VVTAADPASAGRGVLVVMNDTVLDGRDVTKTNT |
| | | TDVATFKSVNYGPLGYIHNGKIDYQRTPARPHT |
| | | LATEFDVSSLTELPKVGIVYNYANASVLPAKAL |
| | | VDAGYDGIVSAGVGNGNLYKEVFDTLAKAAEDG |
| | | TAVVRSSRVPTGATTQDAEVDDAKYGFVASGTL |
| | | NPQKARVLLQLALTKTKDPEEIQKIFDTY |
| V4 (SEQ- | No: 7 | IKKITILATGGTIAGGGDSATKSNYTVGKVGVE |
| 6096) | | ELVERVPQLKDIAEVKGEQVMNIGSQDMNDEVW |
| | | LKLAKKINEDCDKTDGFVITHGTDTMEETAYFL |
| | | DLTVKCDKPVVMVGAMRPSTSMSADGPFNLYNA |
| | | VVTAADQRSAGRGVLVVMNDTVLDGRDVTKTNT |
| | | TDVATFKSVNYGPLGYIHNGKIDYQRTPARPHT |
| | | PDTEFDVSKLTELPKVGIVYNYANASSLPAKAL |
| | | VEAGYDGIVSAGVGNGNLYKSVFDTLAKAAEDG |
| | | TAVVRSSRVPTGATTQDAEVDDAKYGFVASGTL |
| | | NPQKARVLLQLALTKTKDPEEIQRIFDTY |
| V5 (SEQ- | No: 8 | IKNITILATGGTIAGGGDSATKSNYTVGKVGVE |
| 6098) | | NLVKAVPQLKDIANVRGEQVKNIGSQDMNDEVW |
| | | LKLAEKINKDCDKTDGFVITHGTDTMEETAYFL |
| | | DLTVKCDKPVVMVGAMRPSTSMSADGPFNLYNA |
| | | VVTAADQRSAGRGVLVVMNDTVLDGRDVTKTNT |
| | | TDVATFKSVNYGPLGYIHNGKIDYQRTPARLHT |
| | | PLTEFDVSSLDALPKVGIVYNYANASALPAKAL |
| | | VDAGYDGIVSAGVGNGNLYKEVFQTLAQAAADG |
| | | TAVVRSSRVPTGATTQDAEVDDAKYGFVASGTL |
| | | NPQKARVLLQLALTKTKDPEEIQEIFDRY |
| V6 (SEQ- | No: 9 | LPNITILATGGTIAGGGDSATKSNYTVGKVGVD |
| 6099) | | NLVALVPQLKDIANVKGEQVKNIGSQDMNDEVW |
| | | LKLAKKINDDCDKTDGFVITHGTDTMEETAYFL |
| | | DLTVKCDKPVVMVGAMRPSTSMSADGPFNLYNA |
| | | VVTAADERSAGRGVLVVMNDTVLDGRDVTKTNT |
| | | TDVATFKSVNYGPLGYIHNGKIDYQRTPARKHT |
| | | PLTEFDVSNLTELPKVGIVYNYANASDLPAKAL |
| | | VDAGYDGIVSAGVGNGNLYKLVFITLATAAKNG |
| | | TAVVRSSRVPTGATTQDAEVDDEKYGFVASGTL |
| | | NPQKARVLLQLALTKTNDPREIQEIFDTY |
| V7 (SEQ- | No: 10 | LPNITILATGGTIAGGGDSATKSNYTVGKVGVE |
| 6101) | | NLVKAVPQLKDIANVKGEQVKNIGSQDMNDNVW |
| | | LRLAKKINDDCDKTDGFVITHGTDTMEETAYFL |
| | | DLTVKCDKPVVMVGAMRPSTSMSADGPFNLYNA |
| | | VVTAADKRSAGRGVLVVMNDTVLDGRDVTKTNT |
| | | TDVATFKSVNYGPLGYIHNGKIDYQRTPARKHT |
| | | PLTPFDVSKLNELPKVGIVYNYANASSLPAKAL |
| | | VDAGYDGIVSAGVGNGNLYKSVFDTLATAAKNG |
| | | TAVVRSSRVPTGATTQDAEVDDAKYGFVASGTL |
| | | NPQKARVLLQLALTKTNDPQQIQQIFNQY |
| V8 (SEQ- | No: 11 | KKKITILATGGTIAGGGDSATKSNYTVGKVGVD |
| 6093) | | ELIARVPQLADIAEVDGEQVMNIGSQDMNDEVW |
| | | LKLAKRINELCEQYDGFVITHGTDTMEETAYFL |
| | | DLTVKCSKPVVMVGAMRPSTSMSADGPFNLYNA |
| | | VVTAAHPESKGQGVLVVMNDTVLDGRDVTKTNT |
| | | TDVATFKSVNYGPLGYIHNGKIDYQRQPARPHT |
| | | LASEFDVSKIEELPKVGIVYNYANAFVLPAEAL |
| | | IKAGYDGIVSAGVGNGNLYKEVFKALKEAAKNG |
| | | IAVVRSSRVPTGATTQDAEVDDEQYGFVASGTL |
| | | NPQKARVLLQLALVKTNDRREIQRIFDTY |
| V9 (SEQ- | No: 12 | LPNITILATGGTIAGGGDSATKSNYTVGKVGVE |
| 6100) | | NLVAAVPQLKDIANVKVEQVKNIGSQDMNDEVW |
| | | LKLAKKINDDCDKTDGFVITHGTDTMEETAYFL |
| | | DLTVKCDKPVVMVGAMRPSTSMSADGPFNLYNA |
| | | VVTAADKRSANRGVLVVMNDTVLDGRDVTKTNT |
| | | TDVATFKSVNYGPLGYIHNGKIDYQRTPARLHT |
| | | LETEFDVSKLDELPKVGIVYNYANASVLPAKAL |
| | | VDAGYDGIVSAGVGNGNLYKEVFKTLAEAARRG |
| | | TAVVRSSRVPTGATTQDAEVDDEKYGFVASGTL |
| | | NPQKARVLLQLALTKTNDPREIQEIFRRY |
| V10 (SEQ- | No: 13 | MKKITILATGGTIAGGGDSATKSNYTVGKVGVD |
| 6103) | | ELVAGVPQLADIADVTAEEVRNIGSQDMNDEVW |
| | | LELARKINEDCEKTDGFVITHGTDTMEETAYFL |
| | | DLTVTCNKPVVMVGAMRPSTSMSADGPFNLYNA |
| | | VVTAADDASAGRGVLVVMNDTVLDGRDVTKTNT |
| | | TDVATFKSVNYGPLGYIHNGKIDYQRTPARKHT |
| | | KDTPFDVSGLTSLPKVGIVYNYANASSLPAKAL |
| | | VEAGYDGIVSAGVGNGNLYKEVFETLAEAAKNG |
| | | TAVVRSSRVPTGATTQDAEVDDAAYGFVASGTL |
| | | NPQKARVLLQLALTLTKDPAAIQAIFDEY |

Leader Sequence

| | | |
|---|---|---|
| E. coli | No: 14 | MEFFKKTALAALVMGFSGAALAGS |
| Erwinia | No: 15 | MERWFKSLFVLVLFFVFTASAADGS |
| Resur- | No: 16 | MKYLLPTAAAGLLLLAAQPAMAGS |
| faced | | |
| 6x His | No: 17 | HHHHHH |
| tag | | |
| TEV | No: 18 | ENLYFQG |
| protease | | |
| cleavage | | |
| site | | |

What is claimed is:

1. A de-immunized asparaginase polypeptide that, upon administration to a mammalian subject, elicits a reduced immunogenicity in the subject, relative to a bacterial asparaginase.

2. The polypeptide of claim 1, wherein the polypeptide comprises an amino acid sequence that is at least about 30% identical to the amino acid sequence of E. coli asparaginase (SEQ ID NO:1), Erwinia asparaginase (SEQ ID NO:2), or a combination thereof.

3. The polypeptide of claim 2, wherein the polypeptide comprises an amino acid sequence that is at least about 75% identical to the amino acid sequence of E. coli asparaginase (SEQ ID NO:1).

4. The polypeptide of claim 1, wherein the polypeptide comprises at least one amino acid substitution, relative to the amino acid sequence of E. coli asparaginase (SEQ ID NO:1), Erwinia asparaginase (SEQ ID NO:2), or a combination thereof.

5. The polypeptide of claim 4, wherein the polypeptide comprises about 5-60 amino acid substitutions, relative to the amino acid sequence of E. coli asparaginase (SEQ ID NO:1).

6. The polypeptide of claim 4 or 5, wherein the amino acid substitutions include at least one conservative substitution.

7. The polypeptide of claim 4 or 5, wherein the amino acid substitutions include at least one highly conservative substitution.

8. The polypeptide of any one of claims 5-7, wherein the polypeptide comprises an amino acid substitution at a position corresponding to one or more of positions 1, 2, 3, 33, 34, 36, 37, 38, 43, 47, 49, 50, 52, 54, 64, 68, 71, 72, 75, 76, 78, 79, 80, 104, 106, 138, 139, 140, 142, 143, 144, 192, 196, 199, 200, 201, 202, 207, 208, 209, 210, 224, 225, 229, 232, 233, 252, 255, 256, 258, 259, 262, 263, 265, 287, 288, 311, 312, 314, 316, 317, 318, 321, 324 or 325 of SEQ ID NO:1.

9. The polypeptide of claim 8, wherein the polypeptide comprises an amino acid substitution at a position corresponding to one or more of positions 37, 54, 64, 68, 75, 140, 143, 199, 201, 202, 225, 232, 252, 255, 259, 263, 311, 312, 314, 317, 318, 321, 324, or 325 of SEQ ID NO:1.

10. The polypeptide of claim 9, wherein the polypeptide comprises an amino acid substitution at a position corresponding to one or more of positions 54, 64, 68, 75, 143, 199, 201, 202, 232, 263, 311, 312, 317, 318 or 324 of SEQ ID NO:1.

11. The polypeptide of claim 5, wherein the polypeptide comprises an amino acid substitution at a position corresponding to one or more of positions:
a) 54, 201, 202, 232, 263 or 311 of SEQ ID NO:1;
b) 2, 37, 43, 54, 64, 68, 75, 139, 140, 143, 196, 199, 202, 209, 210, 225, 252, 255, 259, 263, 312, 314, 317, 318, 321, 324 or 325 of SEQ ID NO:1;
c) 2, 33, 34, 37, 54, 64, 68, 71, 139, 143, 196, 199, 200, 202, 207, 209, 225, 252, 259, 262, 263, 312, 317, 318, 321, 324 or 325 of SEQ ID NO:1;
d) 1, 2, 3, 34, 37, 38, 47, 54, 64, 68, 75, 139, 140, 143, 196, 199, 202, 209, 225, 233, 259, 262, 263, 312, 317, 318, 321, 324 or 325 of SEQ ID NO:1;
e) 1, 2, 37, 49, 54, 64, 68, 71, 75, 139, 140, 143, 196, 199, 200, 202, 207, 209, 210, 225, 252, 255, 259, 262, 263, 312, 317, 318, 321, 324, 325 of SEQ ID NO:1;
f) 33, 37, 38, 54, 64, 68, 75, 139, 140, 143, 199, 200, 202, 207, 209, 252, 255, 263, 287, 312, 314, 317, 318, 321, 324, 325 of SEQ ID NO:1;
g) 37, 54, 68, 75, 140, 143, 199, 200, 225, 263, 312 or 314 of SEQ ID NO:1;
h) 1, 2, 3, 33, 34, 36, 37, 38, 43, 47, 49, 54, 64, 68, 72, 75, 76, 78, 79, 80, 106, 138, 139, 140, 142, 143, 144, 192, 196, 199, 200, 201, 202, 208, 209, 224, 225, 229, 232, 233, 252, 255, 256, 258, 259, 263, 265, 287, 288, 311, 312, 314, 316, 317, 318, 321, 324 or 325 of SEQ ID NO:1;
i) 37, 50, 54, 64, 68, 75, 140, 196, 199, 200, 202, 209, 225, 252, 255, 259, 262, 263, 287, 312, 314, 317, 318, 321, 324 or 325 of SEQ ID NO:1; or
j) 1, 2, 3, 33, 34, 37, 38, 43, 47, 49, 50, 52, 54, 64, 68, 71, 75, 78, 104, 106, 139, 143, 199, 207, 209, 210, 225, 233, 252, 255, 259, 263, 288, 312, 317, 318, 321, 324 or 325 of SEQ ID NO:1.

12. The polypeptide of claim 5, comprising an amino acid substitution at a position corresponding to the position in SEQ ID NO:1 selected from: L1I, L1K or L1M; P2K; N3K; E33D; N34E or N34K; V36I; N37A, N37E or N37K; A38G, A38L or A38R; K43A; N47D or N47E; K49D, K49R or K49T; G50A or G50V; Q52E; V54K, V54M or V54R; N64E; T68E, T68K or T68R; K71E, K71L or K71R; K72R; T75D, T75E or T75K; D76L; D78E; K79Q; T80Y; K104T; D106N or D106S; D138H; K139D, K139E, K139P or K139Q; A140E or A140R; A142K; N143G; R144Q; T192Q; K196L or K196P; S199K, S199L or S199P; D200A, D200E or D200L; T201S; P202E; K207G, K207N or K207S; L208I; N209D, N209E or N209T; E210A or E210S; S224F; D225A, D225S or D225V; K229E; V232I; D233E or D233K; S252E or S252L; D255E, D255I, D255K or D255Q; T256A; A258K; T259E, T259K or T259Q; K262A, K262E or K262R; T263D, T263N or T263R; T265I; A287E; K288A or K288Q; T311I or T311V; Q312K or Q312L; K314N; P316R; Q317A, Q317E or Q317R; Q318A or Q318E; Q321A, Q321E, Q321K or Q321R; N324D or N324R; Q325E, Q325R or Q325T of SEQ ID NO:1, or a combination thereof.

13. The polypeptide of claim 12, comprising an amino acid substitution at a position corresponding to the position in SEQ ID NO: 1 selected from: L1I, L1K or LIM; P2K; N3K; E33D; N34E or N34K; N37A, N37E or N37K; A38G, A38L or A38R; K43A; N47D or N47E; K49D, K49R or K49T; G50A or G50V; V54K, V54M or V54R; N64E; T68E, T68K or T68R; K71E, K71L or K71R; T75D, T75E or T75K; D78E; D106N or D106S; D138H; K139D, K139E, K139P or K139Q; A140E or A140R; N143G; K196L or K196P; S199K, S199L or S199P; D200A, D200E or D200L; T201S; P202E; K207G, K207N or K207S; N209D, N209E or N209T; E210A or E210S; D225A, D225S or D225V; V232I; D233E or D233K; S252E or S252L; D255E, D255I, D255K or D255Q; T259E, T259K or T259Q; K262A, K262E or K262R; T263D, T263N or T263R; A287E; K288A or K288Q; T311I or T311V; Q312K or Q312L; K314N; Q317A, Q317E or Q317R; Q318A or Q318E; Q321A, Q321E, Q321K or Q321R; N324D or N324R; Q325E, Q325R or Q325T of SEQ ID NO: 1, or a combination thereof.

14. The polypeptide of claim 12, comprising an amino acid substitution at a position corresponding to the position in SEQ ID NO:1 selected from: N37A, N37E or N37K; V54K or V54R; N64E; T68E, T68K or T68R; T75D, T75E or T75K; A140E or A140R; N143G; S199K, S199L or S199P; T201S; P202E; D225A, D225S or D225V; V232I; S252E or S252L; D255E, D255I, D255K or D255Q; T259E, T259K or T259Q; T263D, T263N or T263R; T311I or T311V; Q312K or Q312L; K314N; Q317A, Q317E or Q317R; Q318A or Q318E; Q321A, Q321E, Q321K or Q321R; N324D or N324R; Q325E, Q325R or Q325T, or a combination thereof.

15. The polypeptide of claim 12, comprising an amino acid substitution at a position corresponding to the position in SEQ ID NO:1 selected from: V54K, N64E, T68K, T75E, N143G, S199P, T201S, P202E, V232I, T263D, T311I, Q312K, Q317E, Q318E or N324D, or a combination thereof.

16. The polypeptide of claim 12, comprising an amino acid substitution at a position corresponding to the position in SEQ ID NO:1 selected from:
a) V54K, T201S, P202E, V232I, T263D or T311I, or a combination thereof,
b) P2K, N37K, K43A, V54K, N64E, T68K, T75D, K139Q, A140R, N143G, K196P, S199P, P202E, N209T, E210S, D225V, S252E, D255K, T259E, T263N, Q312K, K314N, Q317E, Q318E, Q321R, N324D or Q325T, or a combination thereof,
c) P2K, E33D, N34K, N37E, V54K, N64E, T68K, K71L, K139P, N143G, K196P, S199L, D200A, P202E, K207S, N209T, D225V, S252E, T259K, K262E, T263D, Q312K, Q317E, Q318E, Q321K, N324D or Q325T, or a combination thereof,
d) L1I, P2K, N3K, N34E, N37E, A38R, N47E, V54M, N64E, T68K, T75E, K139Q, A140R, N143G, K196P, S199P, P202E, N209T, D225S, D233E, T259K, K262E, T263D, Q312K, Q317E, Q318E, Q321R, N324D or Q325T, or a combination thereof,
e) L1I, P2K, N37K, K49R, V54K, N64E, T68K, K71E, T75K, K139Q, A140R, N143G, K196L, S199P, D200L, P202E, K207S, N209D, E210A, D225A, S252E, D255Q, T259Q, K262A, T263D, Q312K, Q317E, Q318E, Q321E, N324D or Q325R, or a combination thereof;
f) E33D, N37A, A38L, V54K, N64E, T68K, T75D, K139E, A140R, N143G, S199P, D200L, P202E, K207N, N209T, S252L, D2551, T263N, A287E, Q312K, K314N, Q317R, Q318E, Q321E, N324D or Q325T, or a combination thereof,
g) N37K, V54K, T68R, T75D, A140R, N143G, S199P, D200L, D225S, T263N, Q312K or K314N, or a combination thereof,
h) L1K, P2K, N3K, E33D, N34E, V36I, N37A, A38R, K43A, N47E, K49D, V54M, N64E, T68K, K72R, T75E, D76L, D78E, K79Q, T80Y, D106S, D138H, K139P, A140E, A142K, N143G, R144Q, T192Q, K196P, S199L, D200A, T201S, P202E, L208I, N209E, S224F, D225V, K229E, V232I, D233K, S252E, D255K, T256A, A258K, T259E, T263N, T265I, A287E, K288Q, T311V, Q312K, K314N, P316R, Q317R, Q318E, Q321R, N324D or Q325T, or a combination thereof,
i) N37A, G50V, V54K, N64E, T68K, T75D, A140R, K196L, S199L, D200E, P202E, N209D, D225V, S252E, D255K, T259E, K262R, T263R, A287E, Q312K, K314N, Q317R, Q318E, Q321E, N324R or Q325R, or a combination thereof; or
j) LIM, P2K, N3K, E33D, N34E, N37A, A38G, K43A, N47D, K49T, G50A, Q52E, V54R, N64E, T68E, K71R, T75E, D78E, K104T, D106N, K139D, N143G, S199K, K207G, N209T, E210S, D225S, D233E, S252E, D255E, T259E, T263N, K288A, Q312L, Q317A, Q318A, Q321A, N324D or Q325E, or a combination thereof.

17. The polypeptide of any one of claims 4-16, wherein the polypeptide comprises a conservative substitution, a highly conservative substitution or no substitution, at the positions corresponding to each of positions 12, 25, 89, 90, 162 and 283 of SEQ ID NO:1.

18. The polypeptide of claim 17, wherein the polypeptide does not comprise a substitution at the positions corresponding to each of positions 12, 25, 89, 90, 162 and 283 of SEQ ID NO:1.

19. The polypeptide of any one of claims 4-18, wherein the polypeptide comprises a conservative substitution, a highly conservative substitution or no substitution, at the positions corresponding to each of positions 23, 41, 60, 63, 94, 121, 122, 123, 151, 156, 163, 164, 165, 167, 175, 176, 181, 184, 186, 218, 222, 248, 250, 251, 272, 275, 277, 281 and 300 of SEQ ID NO:1.

20. The polypeptide of claim 19, wherein the polypeptide does not comprise a substitution at the positions corresponding to each of positions 23, 41, 60, 63, 94, 121, 122, 123, 151, 156, 163, 164, 165, 167, 175, 176, 181, 184, 186, 218, 222, 248, 250, 251, 272, 275, 277, 281 and 300 of SEQ ID NO:1.

21. The polypeptide of any one of claims 4-20, wherein the polypeptide does not comprise a T→V or T→Q substitution at the position corresponding to the T residue at position 192 of SEQ ID NO:1.

22. The polypeptide of claim 21, wherein the polypeptide does not comprise a substitution at the position corresponding to position 192 of SEQ ID NO:1.

23. The polypeptide of any one of claims 4-22, wherein the polypeptide does not comprise a substitution of the T residue at the position corresponding to position 80 of SEQ ID NO:1 with an aromatic amino acid.

24. The polypeptide of claim 23, wherein the polypeptide does not comprise a substitution at the position corresponding to position 80 of SEQ ID NO: 1.

25. The polypeptide of any one of claims 2-24, wherein the polypeptide further comprises a truncation of 1-20 amino acids corresponding to the amino acids at positions 1-20 of E. coli asparaginase (SEQ ID NO:1).

26. The polypeptide of any one of claims 2-25, wherein the polypeptide further comprises a truncation of 1-20 amino acids corresponding to the amino acids at positions 306-326 of E. coli asparaginase (SEQ ID NO:1).

27. The polypeptide of claim 1, wherein the polypeptide comprises an amino acid sequence set forth in any one of SEQ ID Nos:4-13, or a variant thereof comprising an amino acid sequence that is at least 50% identical to the amino acid sequence of any one of SEQ ID Nos:4-13, or a combination thereof.

28. The polypeptide of claim 27, wherein the polypeptide comprises an amino acid sequence that is at least 75% identical to the amino acid sequence of any one of SEQ ID Nos:4-13, or a combination thereof.

29. The polypeptide of claim 27, wherein the polypeptide comprises one or more amino acid substitutions, relative to the amino acid sequence of any one of SEQ ID Nos:4-13, or a combination thereof.

30. The polypeptide of claim 29, wherein the polypeptide comprises about 5-60 amino acid substitutions, relative to the amino acid sequence of any one of SEQ ID Nos:4-13, or a combination thereof.

31. The polypeptide of claim 29 or 30, wherein the amino acid substitutions include at least one conservative substitution.

32. The polypeptide of claim 29 or 30, wherein the amino acid substitutions include at least one highly conservative substitution.

33. The polypeptide of claim 27, wherein the polypeptide comprises the amino acid sequence of SEQ ID NO:4, SEQ ID NO:5, SEQ ID NO:6, SEQ ID NO:7, SEQ ID NO: 8, SEQ ID NO:9, SEQ ID NO:10, SEQ ID NO:11, SEQ ID NO:12 or SEQ ID NO: 13.

34. The polypeptide of claim 33, wherein the polypeptide comprises the amino acid sequence of SEQ ID NO:8, SEQ ID NO:9, SEQ ID NO:11 or SEQ ID NO:13.

35. The polypeptide of any one of claims 1-34, further comprising a leader sequence, a peptide tag sequence or a TEV protease cleavage site, or a combination thereof.

36. The polypeptide of claim 35, wherein:
a) the leader sequence is set forth in SEQ ID NO:14, SEQ ID NO:15 or SEQ ID NO:16;
b) the peptide tag sequence is set forth in SEQ ID NO: 17;
c) the TEV protease cleavage site is set forth in SEQ ID NO:18,
or a combination thereof.

37. The polypeptide of any one of claims 1-36, wherein the polypeptide is conjugated to a heterologous moiety.

38. The polypeptide of claim 37, wherein the heterologous moiety is a therapeutic agent, a diagnostic agent or a combination thereof.

39. The polypeptide of claim 37, wherein the heterologous moiety is selected from the group consisting of polyethylene glycol (PEG), hexadecanoic acid, a hydrogel, a lipid nanoparticle, a polymer nanoparticle, and a heterologous polypeptide sequence, or a combination thereof.

40. The polypeptide of claim 39, wherein the polymer nanoparticle comprises poly(lactic-co-glycolic) acid (PLGA).

41. The polypeptide of claim 37, wherein the heterologous polypeptide sequence comprises a carrier polypeptide.

42. The polypeptide of claim 41, wherein the carrier polypeptide is albumin or an Fc polypeptide.

43. The polypeptide of any one of claims 1-42, wherein the polypeptide:
a) can be produced with an overall yield of purified asparaginase of at least about 1 mg asparaginase per liter of E. coli culture;
b) can be produced with an enzyme yield of at least about 50%, relative to E. coli asparaginase (SEQ ID NO:1);
c) binds an anti-asparaginase antibody with an affinity that is at least 10% lower, relative to the E. coli asparaginase (SEQ ID NO: 1),
d) or any combination thereof.

44. A fusion protein comprising the polypeptide of any one of claims 1-43.

45. A polynucleotide comprising a sequence encoding the polypeptide of any one of claims 1-43 or the fusion protein of claim 44.

46. An expression vector comprising the polynucleotide of claim 45.

47. A host cell comprising the polynucleotide of claim 45 or the expression vector of claim 46.

48. A composition comprising the polypeptide of any one of claims 1-43, the fusion protein of claim 44, the polynucleotide of claim 45, the expression vector of claim 46 or the host cell of claim 47.

49. The composition of claim 48, comprising the polypeptide of any one of claims 1-43 or the fusion protein of claim 43.

50. The composition of claim 48 or 49, further comprising one or more pharmaceutically acceptable excipients, diluents, or carriers.

51. A method of reducing a level of asparagine or asparagine-containing product in a biological fluid from a mammalian subject, comprising contacting the biological fluid with an effective amount of the polypeptide of any one of claims 1-43, the fusion protein of claim 44, or the composition of any one of claims 48-50.

52. A method of treating a mammalian subject in need thereof, comprising administering an effective amount of the polypeptide of any one of claims 1-43, the fusion protein of claim 44, or the composition of any one of claims 48-50 to the mammalian subject.

53. The method of claim 51 or 52, wherein the mammalian subject has cancer.

54. A method of treating cancer in a mammalian subject in need thereof, comprising administering an effective amount of the polypeptide of any one of claims 1-43, the fusion protein of claim 44, or the composition of any one of claims 48-50 to the mammalian subject.

55. The method of claim 53 or 54, wherein the cancer is a hematological cancer.

56. The method of claim 55, wherein the hematological cancer is acute lymphoblastic leukemia (ALL), non-Hodgkin's lymphoma or acute myeloid leukemia (AML).

57. The method of any one of claims 51-56, wherein the mammalian subject has an immune response to one or more bacterial asparaginases.

58. The method of any one of claims 51-56, wherein the mammalian subject is at risk of developing an immune response to one or more bacterial asparaginases.

59. The method of claim 57 or 58, wherein the immune response comprises a response mediated by an antibody that specifically binds the one or more bacterial asparaginases.

60. The method of any one of claims 51-59, wherein the mammalian subject is a human.

61. The method of any one of claims 51-60, further comprising administering a second therapeutic agent to the mammalian subject.

62. The method of claim 61, wherein the second therapeutic agent comprises a chemotherapeutic agent, a steroid, or a combination thereof.

63. The method of claim 61 or 62, wherein the composition and the second therapeutic agent are administered concurrently.

64. The method of claim 61 or 62, wherein the composition and the second therapeutic agent are administered sequentially.


SEQUENCE LISTING (18 sequences)

SEQ ID NO: 1 - protein, 326 aa, Escherichia coli
LPNITILATGGTIAGGGDSATKSNYTVGKVGVENLVNAVPQLKDIANVKGEQVVNIGSQDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKSVFDTLATAAKTGTAVVRSSRVPTGATTQDAEVDDAKYGFVASGTLNPQKARVLLQLALTQTKDPQQIQQIFNQY

SEQ ID NO: 2 - protein, 327 aa, Erwinia chrysanthemi
ADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 3 - protein, 326 aa, Artificial Sequence
  Consensus asparaginase sequence
  misc_feature (1)..(3): Xaa can be any naturally occurring amino acid
  misc_feature (33)..(34): Xaa can be any naturally occurring amino acid
  misc_feature (36)..(38): Xaa can be any naturally occurring amino acid
  misc_feature (43)..(43): Xaa can be any naturally occurring amino acid
  misc_feature (47)..(47): Xaa can be any naturally occurring amino acid
  misc_feature (49)..(50): Xaa can be any naturally occurring amino acid
  misc_feature (52)..(52): Xaa can be any naturally occurring amino acid
  misc_feature (54)..(54): Xaa can be any naturally occurring amino acid
  misc_feature (64)..(64): Xaa can be any naturally occurring amino acid
  misc_feature (68)..(68): Xaa can be any naturally occurring amino acid
  misc_feature (71)..(72): Xaa can be any naturally occurring amino acid
  misc_feature (75)..(76): Xaa can be any naturally occurring amino acid
  misc_feature (78)..(80): Xaa can be any naturally occurring amino acid
  misc_feature (104)..(104): Xaa can be any naturally occurring amino acid
  misc_feature (106)..(106): Xaa can be any naturally occurring amino acid
  misc_feature (138)..(140): Xaa can be any naturally occurring amino acid
  misc_feature (142)..(144): Xaa can be any naturally occurring amino acid
  misc_feature (192)..(192): Xaa can be any naturally occurring amino acid
  misc_feature (196)..(196): Xaa can be any naturally occurring amino acid
  misc_feature (199)..(202): Xaa can be any naturally occurring amino acid
  misc_feature (207)..(210): Xaa can be any naturally occurring amino acid
  misc_feature (224)..(225): Xaa can be any naturally occurring amino acid
  misc_feature (229)..(229): Xaa can be any naturally occurring amino acid
  misc_feature (232)..(233): Xaa can be any naturally occurring amino acid
  misc_feature (252)..(252): Xaa can be any naturally occurring amino acid
  misc_feature (255)..(256): Xaa can be any naturally occurring amino acid
  misc_feature (258)..(259): Xaa can be any naturally occurring amino acid
  misc_feature (262)..(263): Xaa can be any naturally occurring amino acid
  misc_feature (265)..(265): Xaa can be any naturally occurring amino acid
  misc_feature (287)..(288): Xaa can be any naturally occurring amino acid
  misc_feature (311)..(312): Xaa can be any naturally occurring amino acid
  misc_feature (314)..(314): Xaa can be any naturally occurring amino acid
  misc_feature (316)..(318): Xaa can be any naturally occurring amino acid
  misc_feature (321)..(321): Xaa can be any naturally occurring amino acid
  misc_feature (324)..(325): Xaa can be any naturally occurring amino acid
XXXITILATGGTIAGGGDSATKSNYTVGKVGVXXLXXXVPQLXDIAXVXXEXVXNIGSQDMNDXVWLXLAXXINXXCXXXDGFVITHGTDTMEETAYFLDLTVXCXKPVVMVGAMRPSTSMSADGPFNLYNAVVTAAXXXSXXXGVLVVMNDTVLDGRDVTKTNTTDVATFKSVNYGPLGYIHNGKIDYQRXPARXHTXXXXFDVSXXXXLPKVGIVYNYANAXXLPAXALXXAGYDGIVSAGVGNGNLYKXVFXXLXXAAXXGXAVVRSSRVPTGATTQDAEVDDXXYGFVASGTLNPQKARVLLQLALXXTXDXXXIQXIFXXY

SEQ ID NO: 4 - protein, 326 aa, Artificial Sequence
  V1
LPNITILATGGTIAGGGDSATKSNYTVGKVGVENLVNAVPQLKDIANVKGEQVKNIGSQDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDSEFDVSKLNELPKVGIVYNYANASDLPAKALIDAGYDGIVSAGVGNGNLYKSVFDTLATAAKDGTAVVRSSRVPTGATTQDAEVDDAKYGFVASGTLNPQKARVLLQLALIQTKDPQQIQQIFNQY

SEQ ID NO: 5 - protein, 326 aa, Artificial Sequence
  V2
LKNITILATGGTIAGGGDSATKSNYTVGKVGVENLVKAVPQLADIANVKGEQVKNIGSQDMNDEVWLKLAKKINDDCDKTDGFVITHGTDTMEETAYFLDLTVKCDKPVVMVGAMRPSTSMSADGPFNLYNAVVTAADQRSAGRGVLVVMNDTVLDGRDVTKTNTTDVATFKSVNYGPLGYIHNGKIDYQRTPARPHTPDTEFDVSKLTSLPKVGIVYNYANASVLPAKALVDAGYDGIVSAGVGNGNLYKEVFKTLAEAAKNGTAVVRSSRVPTGATTQDAEVDDAKYGFVASGTLNPQKARVLLQLALTKTNDPEEIQRIFDTY

SEQ ID NO: 6 - protein, 326 aa, Artificial Sequence
  V3
LKNITILATGGTIAGGGDSATKSNYTVGKVGVDKLVEAVPQLKDIANVKGEQVKNIGSQDMNDEVWLKLALKINTDCDKTDGFVITHGTDTMEETAYFLDLTVKCDKPVVMVGAMRPSTSMSADGPFNLYNAVVTAADPASAGRGVLVVMNDTVLDGRDVTKTNTTDVATFKSVNYGPLGYIHNGKIDYQRTPARPHTLATEFDVSSLTELPKVGIVYNYANASVLPAKALVDAGYDGIVSAGVGNGNLYKEVFDTLAKAAEDGTAVVRSSRVPTGATTQDAEVDDAKYGFVASGTLNPQKARVLLQLALTKTKDPEEIQKIFDTY

SEQ ID NO: 7 - protein, 326 aa, Artificial Sequence
  V4
IKKITILATGGTIAGGGDSATKSNYTVGKVGVEELVERVPQLKDIAEVKGEQVMNIGSQDMNDEVWLKLAKKINEDCDKTDGFVITHGTDTMEETAYFLDLTVKCDKPVVMVGAMRPSTSMSADGPFNLYNAVVTAADQRSAGRGVLVVMNDTVLDGRDVTKTNTTDVATFKSVNYGPLGYIHNGKIDYQRTPARPHTPDTEFDVSKLTELPKVGIVYNYANASSLPAKALVEAGYDGIVSAGVGNGNLYKSVFDTLAKAAEDGTAVVRSSRVPTGATTQDAEVDDAKYGFVASGTLNPQKARVLLQLALTKTKDPEEIQRIFDTY

SEQ ID NO: 8 - protein, 326 aa, Artificial Sequence
  V5
IKNITILATGGTIAGGGDSATKSNYTVGKVGVENLVKAVPQLKDIANVRGEQVKNIGSQDMNDEVWLKLAEKINKDCDKTDGFVITHGTDTMEETAYFLDLTVKCDKPVVMVGAMRPSTSMSADGPFNLYNAVVTAADQRSAGRGVLVVMNDTVLDGRDVTKTNTTDVATFKSVNYGPLGYIHNGKIDYQRTPARLHTPLTEFDVSSLDALPKVGIVYNYANASALPAKALVDAGYDGIVSAGVGNGNLYKEVFQTLAQAAADGTAVVRSSRVPTGATTQDAEVDDAKYGFVASGTLNPQKARVLLQLALTKTKDPEEIQEIFDRY

SEQ ID NO: 9 - protein, 326 aa, Artificial Sequence
  V6
LPNITILATGGTIAGGGDSATKSNYTVGKVGVDNLVALVPQLKDIANVKGEQVKNIGSQDMNDEVWLKLAKKINDDCDKTDGFVITHGTDTMEETAYFLDLTVKCDKPVVMVGAMRPSTSMSADGPFNLYNAVVTAADERSAGRGVLVVMNDTVLDGRDVTKTNTTDVATFKSVNYGPLGYIHNGKIDYQRTPARKHTPLTEFDVSNLTELPKVGIVYNYANASDLPAKALVDAGYDGIVSAGVGNGNLYKLVFITLATAAKNGTAVVRSSRVPTGATTQDAEVDDEKYGFVASGTLNPQKARVLLQLALTKTNDPREIQEIFDTY

SEQ ID NO: 10 - protein, 326 aa, Artificial Sequence
  V7
LPNITILATGGTIAGGGDSATKSNYTVGKVGVENLVKAVPQLKDIANVKGEQVKNIGSQDMNDNVWLRLAKKINDDCDKTDGFVITHGTDTMEETAYFLDLTVKCDKPVVMVGAMRPSTSMSADGPFNLYNAVVTAADKRSAGRGVLVVMNDTVLDGRDVTKTNTTDVATFKSVNYGPLGYIHNGKIDYQRTPARKHTPLTPFDVSKLNELPKVGIVYNYANASSLPAKALVDAGYDGIVSAGVGNGNLYKSVFDTLATAAKNGTAVVRSSRVPTGATTQDAEVDDAKYGFVASGTLNPQKARVLLQLALTKTNDPQQIQQIFNQY

SEQ ID NO: 11 - protein, 326 aa, Artificial Sequence
  V8
KKKITILATGGTIAGGGDSATKSNYTVGKVGVDELIARVPQLADIAEVDGEQVMNIGSQDMNDEVWLKLAKRINELCEQYDGFVITHGTDTMEETAYFLDLTVKCSKPVVMVGAMRPSTSMSADGPFNLYNAVVTAAHPESKGQGVLVVMNDTVLDGRDVTKTNTTDVATFKSVNYGPLGYIHNGKIDYQRQPARPHTLASEFDVSKIEELPKVGIVYNYANAFVLPAEALIKAGYDGIVSAGVGNGNLYKEVFKALKEAAKNGIAVVRSSRVPTGATTQDAEVDDEQYGFVASGTLNPQKARVLLQLALVKTNDRREIQRIFDTY

SEQ ID NO: 12 - protein, 326 aa, Artificial Sequence
  V9
LPNITILATGGTIAGGGDSATKSNYTVGKVGVENLVAAVPQLKDIANVKVEQVKNIGSQDMNDEVWLKLAKKINDDCDKTDGFVITHGTDTMEETAYFLDLTVKCDKPVVMVGAMRPSTSMSADGPFNLYNAVVTAADKRSANRGVLVVMNDTVLDGRDVTKTNTTDVATFKSVNYGPLGYIHNGKIDYQRTPARLHTLETEFDVSKLDELPKVGIVYNYANASVLPAKALVDAGYDGIVSAGVGNGNLYKEVFKTLAEAARRGTAVVRSSRVPTGATTQDAEVDDEKYGFVASGTLNPQKARVLLQLALTKTNDPREIQEIFRRY

SEQ ID NO: 13 - protein, 326 aa, Artificial Sequence
  V10
MKKITILATGGTIAGGGDSATKSNYTVGKVGVDELVAGVPQLADIADVTAEEVRNIGSQDMNDEVWLELARKINEDCEKTDGFVITHGTDTMEETAYFLDLTVTCNKPVVMVGAMRPSTSMSADGPFNLYNAVVTAADDASAGRGVLVVMNDTVLDGRDVTKTNTTDVATFKSVNYGPLGYIHNGKIDYQRTPARKHTKDTPFDVSGLTSLPKVGIVYNYANASSLPAKALVEAGYDGIVSAGVGNGNLYKEVFETLAEAAKNGTAVVRSSRVPTGATTQDAEVDDAAYGFVASGTLNPQKARVLLQLALTLTKDPAAIQAIFDEY

SEQ ID NO: 14 - protein, 24 aa, Escherichia coli
MEFFKKTALAALVMGFSGAALAGS

SEQ ID NO: 15 - protein, 25 aa, Erwinia chrysanthemi
MERWFKSLFVLVLFFVFTASAADGS

SEQ ID NO: 16 - protein, 24 aa, Artificial Sequence
  Resurfaced
MKYLLPTAAAGLLLLAAQPAMAGS

SEQ ID NO: 17 - protein, 6 aa, Artificial Sequence
  6x His tag
HHHHHH

SEQ ID NO: 18 - protein, 7 aa, Artificial Sequence
  TEV protease cleavage site
ENLYFQG